(19) United States
(12) Patent Application Publication
(10) Pub. No.: US 2026/0041767 A1
(43) Pub. Date: Feb. 12, 2026

(54) METHODS OF IMPROVING CELLULAR THERAPY WITH ORGANELLE COMPLEXES

(71) Applicants: National University Corporation Hokkaido University, Sapporo-shi, Hokkaido (JP); LUCA Science Inc., Tokyo (JP)

(72) Inventors: Takanori Teshima, Sapporo-shi, Hokkaido (JP); Daigo Hashimoto, Sapporo-shi, Hokkaido (JP); Masashi Suganuma, Tokyo (JP); Rick C. Tsai, Tokyo (JP)

(21) Appl. No.: 19/100,317

(22) PCT Filed: Aug. 1, 2023
(86) PCT No.: PCT/US2023/029228

§ 371 Date: Jan. 31, 2025

Related U.S. Application Data
(60) Provisional application No. 63/394,546, filed on Aug. 2, 2022.
(60) Provisional application No. 63/403,268, filed on Sep. 1, 2022.
(60) Provisional application No. 63/406,022, filed on Sep. 13, 2022.

(51) Int. Cl. A61K 40/11; A61K 31/7088; A61K 35/17; A61K 38/17; A61K 40/31; A61K 40/32; A61K 40/42; A61P 35/00; C07K 14/725; C12N 5/0783; C12N 5/10

(52) U.S. Cl. CPC A61K 40/11; A61K 31/7088; A61K 35/17; A61K 38/1774; A61K 40/31; A61K 40/32; A61K 40/42; A61P 35/00; C07K 14/7051; C12N 5/0637; C12N 5/0638; C12N 5/10; A61K 2239/48; C12N 2501/998; C12N 2510/00

(57) ABSTRACT

Disclosed herein include methods, compositions, and kits suitable for use in enhancing adoptive T cell therapy. In some embodiments, the method comprises contacting isolated organelle complexes with a population of T cells to generate a population of T cells comprising the organelle complexes. The organelle complexes can comprise mitochondria and one or more of endoplasmic reticulum, peroxisomes, lysosomes, and Golgi apparatus. The population of T cells can exhibit one or more of enhanced expansion capability, enhanced cytotoxicity against target cells, enhanced resistance to exhaustion, and enhanced persistence, as compared to a population of T cells that do not comprise exogenous organelle complexes

### RELATED APPLICATIONS

The present application claims priority to U.S. Provisional Application No. 63/394,546, filed Aug. 2, 2022; U.S. Provisional Application No. 63/403,268, filed Sep. 1, 2022; and U.S. Provisional Application No. 63/406,022, filed Sep. 13, 2022. The entire contents of these applications are hereby expressly incorporated by reference in their entireties.

### BACKGROUND

### Field

The present disclosure relates generally to methods of enhancing the proliferation, migration, persistence and/or activity of T cells for adoptive T cell therapy.

### Description of the Related Art

Mitochondria are intracellular organelles responsible for a number of metabolic transformations and regulatory functions. Mitochondria are also highly dynamic organelles that move throughout the cell and undergo structural transitions, changing the length, morphology, shape and size. Moreover, mitochondria are continuously eliminated and regenerated in a process known as mitochondrial biogenesis. While most mitochondrial genes have been transferred to the nuclear genome, the mitochondria genome still encodes rRNAs, tRNAs, and 13 subunits of the electron transport chain (ETC). Functional communication between the nuclear and mitochondrial genomes is therefore essential for mitochondrial biogenesis, efficient oxidative phosphorylation, and normal health. Mitochondria are also the major source of free radicals and reactive oxygen species (ROS) that cause oxidative stress. Additionally, mitochondria play key roles in intracellular signaling as well as control of cell death, including apoptosis and necrosis.

Chimeric antigen receptor (CAR) T cell therapy is a potentially curative treatment for patients with relapsed or refractory (r/r) hematopoietic malignancies. However, roughly half of patients with r/r B-cell lymphoma experience treatment failure after CAR-T cell therapy, possibly due to poor expansion, limited persistence, and exhaustion of CAR-T cells. Given that metabolic fitness has a central role in regulating T cell functions, manipulating the mitochondrial function is a promising approach to enhance anti-tumor effects of CAR-T cells. There is a need for methods of enhancing the proliferation, migration, persistence and/or activity of T cells for adoptive T cell therapy.

### SUMMARY

Provided herein include methods and compositions solving the above-mentioned problems in the art. For example, the transplantation of isolated organelle complexes into T cells as described herein can metabolically reprogram the T cells and thereby improve the performance of said T cells across multiple parameters both in vitro and in vivo, including cytokine production, proliferation, persistence, and anti-tumor activity. Disclosed herein include methods of producing a population of T cells for adoptive T cell therapy. In some embodiments, the method comprises: contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus. In some embodiments, the method comprises stimulating the population of T cells. In some embodiments, stimulating step expands the population of T cells.

Disclosed herein include methods of enhancing the proliferation, migration, persistence and/or activity of a population of T cells for adoptive T cell therapy. In some embodiments, the method comprises: contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus. The method can comprise: stimulating the population of T cells to expand the population of T cells.

Disclosed herein include methods of generating a population of T cells resistant to exhaustion. In some embodiments, the method comprises: contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus. The method can comprise: stimulating the population of T cells to expand the population of T cells.

In some embodiments, the stimulating is performed before the contacting, after the contacting, and/or during contacting. In some embodiments, the stimulating comprises culturing the population of T cells in the presence of one or more stimulating agents. In some embodiments, the one or more stimulating agents comprise an agent that stimulates a CD3/TCR complex-associated signal and an agent that stimulates a costimulatory molecule on the surface of the T cells. In some embodiments, the one or more stimulating agents comprise: a molecule that binds CD28 (e.g., one or more of an anti-CD28 antibody, CD80, and CD86); and/or a molecule that binds CD3 (e.g., an anti-CD3 antibody). In some embodiments, the stimulating and/or contacting is performed for a period time of at least about 6 hours, 12 hours, 16 hours, 1 day, 2 days, 3 days, 4 days, 5 days, 6 days, or 7 days. In some embodiments, upon contact of the isolated organelle complexes with a population of T cells, the organelle complexes are capable of incorporating into the T cells. In some embodiments, the contacting step is repeated at least 2, 3, or 4 times. In some embodiments, the contacting is performed during one or both of the stimulating step and the introducing step. In some embodiments, the population of T cells is contacted with an effective amount of isolated organelle complexes sufficient to enhance the proliferation, migration, persistence and/or activity of said T cells. In some embodiments, the effective amount comprises about 20 ug of isolated organelle complexes. In some embodiments, the population of T cells is contacted with an effective amount of isolated organelle complexes sufficient to render the said T cells resistant to exhaustion. In some embodiments, the population of T cells is derived from lymph node cells (e.g., purified by a magnetic particle-based enrichment procedure selected from the group consisting of manual MACS®, AutoMACS®, CliniMACS®, EasySep®, and RoboSep®). In some embodiments, the population of T cells comprises one or more of a CD4⁺ cell, a CD8⁺ T cell, a cytotoxic T cell, a terminal effector T cell, an effector T cell, a memory or central memory T cell, a naive T cell, a regulatory T cell, a natural killer T cell, a gamma-delta T cell, a cytokine induced killer (CIK) T cell and a tumor infiltrating lymphocyte (TIL).

In some embodiments, the organelle complexes comprise first organelle complexes, second organelle complexes, or a combination of first organelle complexes and second organelle complexes, wherein the first organelle complexes and second organelle complexes are depleted of cytosolic macromolecules, wherein first organelle complexes are derived from (i) frozen cells; (ii) floating cells; and/or (iii) cells contacted with a surfactant at a concentration at or above the critical micellar concentration (CMC) for the surfactant, and wherein second organelle complexes are derived from (i) adherent cells; and/or (ii) cells contacted with a surfactant at a concentration below the critical micellar concentration (CMC) for the surfactant. In some embodiments, the cytosolic macromolecules comprise cytosolic proteins (e.g., the cytosolic proteins are p70S6K and/or glyceraldehyde 3-phosphate dehydrogenase (GAPDH)), wherein the abundance of one or more cytosolic proteins is depleted by at least about 90% as compared to the cells from which the organelle complexes population are derived. In some embodiments, the organelle complexes comprise: one or more mitochondrial matrix proteins (e.g., mitochondrial transcription factor A (TFAM) and/or citrate synthase (CS)); one or more outer mitochondrial membrane proteins (e.g., outer mitochondrial membrane complex subunit 20 (TOMM20)); one or more lysosome proteins (e.g., lysosomal-associated membrane protein 2 (LAMP2), mannose-6-phosphate receptor (M6PR), and/or lysosomal-associated membrane protein 1 (LAMP1)); one or more peroxisome proteins (e.g., catalase and/or ATP-binding cassette transporter 1, subfamily D, type 3 (ABCD3)); one or more Golgi apparatus proteins (e.g., Golgin-97, Sintaxin-6, TGOLN2/trans-Golgi network protein 2 (TGN46), Golgi matrix protein 130 (GM130), and/or Mannosidase Alpha Class 2A Member 1 (MAN2A1)); and/or one or more endoplasmic reticulum proteins (e.g., Calreticulin and/or Calnexin). In some embodiments, the organelle complexes are derived from cells treated with a mitochondria-activating agent (e.g., resveratrol). In some embodiments, the organelle complexes are derived from cells of a subject different from the subject from which the T cells are derived. In some embodiments, the organelle complexes are derived from cells of the same subject from which the T cells are derived.

In some embodiments, the method comprises introducing a heterologous nucleic acid (e.g., a vector) encoding a chimeric antigen receptor (CAR) and/or an engineered T cell receptor (TCR) into the T cells. In some embodiments, the introducing step is performed before the contacting, after the contacting, and/or during the contacting. In some embodiments, the T cell comprises a chimeric antigen receptor (CAR) and/or an engineered T cell receptor (TCR). In some embodiments, the CAR and/or TCR comprises one or more of an antigen binding domain, a transmembrane domain, and an intracellular signaling domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof. In some embodiments, the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS (CD278), lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100(SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof. In some embodiments, the antigen binding domain binds a tumor antigen (e.g., a solid tumor antigen). In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising anticomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the antigen binding domain is connected to the transmembrane domain by a hinge region. In some embodiments, the transmembrane domain comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRTAM, Ly9(CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the CAR or TCR further comprises a leader peptide. In some embodiments, the TCR further comprises a constant region and/or CDR4.

In some embodiments, T cell recovery efficiency is at least about 5 percent greater as compared to a method that does not comprise contacting isolated organelle complexes with the population of T cells. In some embodiments, T cell recovery efficiency is the ratio of T cells recovered after stimulation (e.g., after about 48 hours of stimulation) to the number of T cells immediately before the start of stimulation. In some embodiments, the population of T cells exhibit one or more of enhanced expansion capability (in vivo and/or in vitro), enhanced cytotoxicity against target cells (in vivo and/or in vitro), enhanced resistance to exhaustion (in vivo and/or in vitro), and enhanced persistence (in vivo and/or in vitro), as compared to a population of T cells that do not comprise exogenous organelle complexes. In some embodiments, the population of T cells exhibit an at least about 1.1-fold increase in basal and/or maximal oxygen consumption rate (in vivo and/or in vitro) as compared to a population of T cells that do not comprise exogenous organelle complexes. In some embodiments, the population of T cells exhibit an at least about 1.1-fold increase in basal and/or maximal glycolytic rate (in vivo and/or in vitro) as compared to a population of T cells that do not comprise exogenous organelle complexes. In some embodiments, the population of T cells exhibit an at least about 1.1-fold increase in glycolytic capacity and/or respiratory capacity (in vivo and/or in vitro) as compared to a population of T cells that do not comprise exogenous organelle complexes. In some embodiments, the population of T cells exhibit an at least about 1.1-fold increase in mitochondrial mass (in vivo and/or in vitro) as compared to a population of T cells that do not comprise exogenous organelle complexes. In some embodiments, the population of T cells exhibit an at least about 1.1-fold increase in cytotoxic activity against target cells (in vivo and/or in vitro) as compared to a population of T cells that do not comprise exogenous organelle complexes. In some embodiments, the population of T cells exhibit an at least about 1.1-fold decrease in the level of one or more exhaustion markers (in vivo and/or in vitro) as compared to a population of T cells that do not comprise exogenous organelle complexes. In some embodiments, the exhaustion markers are selected from the group comprising PD-1, CTLA-4, TIM-3, LAG-3, BTLA, 2B4, CD 160, CD39, VISTA, TIGIT, or any combination thereof. In some embodiments, the population of T cells exhibit an at least about 1.1-fold reduction in levels one or more of cellular ROS (in vivo and/or in vitro), mitochondrial ROS (in vivo and/or in vitro), cellular oxidative stress (in vivo and/or in vitro), and mitochondrial oxidative stress (in vivo and/or in vitro), as compared to a population of T cells that do not comprise exogenous organelle complexes. In some embodiments, the reactive oxygen species comprise superoxide (O₂·⁻), hydroperoxy (HO·₂), hydrogen peroxide (H₂O₂), peroxynitrite (ONOO⁻), hypochlorous acid (HOCl), hypobromous acid (HOBr), hydroxyl radical (HO·), peroxy radical (ROO·), alkoxy radical (RO·), singlet oxygen (¹O₂), lipid peroxides, lipid peroxyradicals or lipid alkoxyl radicals, or any combination thereof. In some embodiments, the population of T cells exhibit an at least about 1.1-fold increase production of one or more of cytokines (in vivo and/or in vitro) as compared to a population of T cells that do not comprise exogenous organelle complexes. In some embodiments, the cytokines comprise interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). In some embodiments, the population of T cells exhibit an at least about 1.1-fold increase in cell proliferation (in vivo and/or in vitro) as compared to a population of T cells that do not comprise exogenous organelle complexes. In some embodiments, the population of T cells exhibit an at least about 1.1-fold increase in cell viability following chronic TCR stimulation (in vivo and/or in vitro) as compared to a population of T cells that do not comprise exogenous organelle complexes.

There are provided, in some embodiments, populations of T cells generated by a method disclosed herein. There are provided, in some embodiments, pharmaceutical compositions. In some embodiments, the pharmaceutical composition comprises: a population of T cells generated by a method disclosed herein; and one or more pharmaceutically acceptable carriers.

There are provided, in some embodiments, populations of T cells for an adoptive T cell therapy wherein the T cells comprise exogenous organelle complexes. In some embodiments, the organelle complexes comprise mitochondria and one or more of endoplasmic reticulum, peroxisomes, lysosomes, and Golgi apparatus. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

Disclosed herein include methods of treating or preventing a disease or disorder in a subject. In some embodiments, the method comprises: administering to the subject an effective amount of the population of T cells generated according to a method disclosed herein, thereby treating or preventing the disease or disorder in the subject. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

Disclosed herein include methods for enhancing adoptive T cell therapy in a subject. In some embodiments, the method comprises: generating an effective amount of T cells comprising exogenous organelle complexes according to a method disclosed herein; and adoptively transferring said T cells to the subject. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

Disclosed herein include methods of treating or preventing a disease or disorder in a subject. In some embodiments, the method comprises: administering to the subject an effective amount of a population of T cells (e.g., T cells comprise a chimeric antigen receptor (CAR) and/or an engineered T cell receptor (TCR)); and administering to the subject an effective amount of isolated organelle complexes, thereby treating or preventing the disease or disorder in the subject. In some embodiments, the isolated organelle complexes are capable of contacting the population of T cells in vivo to generate a population of T cells comprising the organelle complexes. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes. In some embodiments, the subject is administered an effective amount of the population of T cells prior to administering to the subject an effective amount of isolated organelle complexes. In some embodiments, the subject is administered an effective amount of isolated organelle complexes prior to administering to the subject an effective amount of the population of T cells. In some embodiments, the subject is administered an effective amount of the population of T cells and an effective amount of isolated organelle complexes simultaneously.

Disclosed herein include methods of treating or preventing a disease or disorder in a subject. In some embodiments, the method comprises: administering to the subject an effective amount of a heterologous nucleic acid (e.g., a vector, a viral vector) encoding a chimeric antigen receptor (CAR) and/or an engineered T cell receptor (TCR); and administering to the subject an effective amount of isolated organelle complexes, thereby treating or preventing the disease or disorder in the subject. In some embodiments, the heterologous nucleic acid and isolated organelle complexes are capable of contacting T cells of the subject in vivo to generate T cells comprising the organelle complexes and a CAR and/or an engineered TCR. In some embodiments, said T cells exhibit one or more of enhanced expansion capability, enhanced cytotoxicity against target cells, enhanced resistance to exhaustion, and enhanced persistence, as compared to T cells that do not comprise exogenous organelle complexes.

In some embodiments, the in vivo persistence of the population of T cells comprises a period of about 15 days, about 30 days, about 60 days, about 90 days, or about a year. In some embodiments, the population of T cells reduces tumor volume, tumor growth, and/or tumor burden in the subject. In some embodiments, the population of T cells reduces tumor volume in the subject at least about 1.1-fold as compared with the tumor volume in an untreated subject or a subject administered a population of T cells that do not comprise exogenous organelle complexes. In some embodiments, the population of T cells increases overall survival or progression-free survival. In some embodiments, the population of T cells increases overall survival or progression-free survival at least about 1.1-fold as compared with untreated subjects or subjects administered a population of T cells that do not comprise exogenous organelle complexes. In some embodiments, the T cells are autologous to the subject. In some embodiments, the T cells are allogenic to the subject. In some embodiments, the adoptive T cell therapy is a CAR-T cell therapy. In some embodiments, the adoptive T cell therapy is an engineered TCR-T cell therapy. In some embodiments, the adoptive T cell therapy is a tumor infiltrating lymphocyte (TIL) therapy. In some embodiments, the administering comprises administering (e.g., intravenously) at least about 1×10⁶ T cells. In some embodiments, the method comprises repeated administrations of the population of T cells.

In some embodiments, the subject is a mammal. In some embodiments, the disease or disorder is associated with expression of a tumor antigen, and wherein the disease associated with expression of a tumor antigen is selected from the group consisting of a proliferative disease, a precancerous condition, a cancer, and a non-cancer related indication associated with expression of the tumor antigen. In some embodiments, the cancer is selected from the group consisting of colon cancer, rectal cancer, renal-cell carcinoma, liver cancer, small cell or non-small cell carcinoma of the lung, mesothelioma, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell-or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, administering comprises systemic administration (e.g., intravenous, intramuscular, intraperitoneal, or intraarticular), intrathecal administration, intracranial injection, aerosol delivery, nasal delivery, vaginal delivery, rectal delivery, buccal delivery, ocular delivery, local delivery, topical delivery, intracistemal delivery, intraperitoneal delivery, oral delivery, intramuscular injection, intravenous injection, subcutaneous injection, intranodal injection, intratumoral injection, intraperitoneal injection, intradermal injection, or any combination thereof.

### BRIEF DESCRIPTION OF THE DRAWINGS

FIG. 1 depicts data related to the enhanced OXPHOS of Q-treated CAR-T cells compared to vehicle-treated CAR-T cells.

FIG. 2 depicts data related to the suppression of tumor growth in the mice injected with Q-treated CAR-T cells.

FIG. 3 depicts data related to the efficiency of T cell recovery after stimulation following transplantation with second organelle complexes (2^{nd} OC) or vehicle.

FIGS. 4A-4D depict data related to mitochondria mass assessment with MitoTracker Deep Red dye. CD4 T cells (FIGS. 4A-4B) and CD8 T cells (FIGS. 4C-4D) were transplanted with second organelle complexes (2^{nd} OC) or vehicle and assessed at day 4.

FIGS. 5A-5D depict data related to T cell proliferation capacity assessed by a dye dilution assay using Cell Trace Violet dye. CD4 T cells (FIGS. 5A-5B) and CD8 T cells (FIGS. 5C-5D) were transplanted with second organelle complexes (2^{nd} OC) or vehicle and assessed at day 4.

FIGS. 6A-6D depict data related to mitochondria-selective oxidative stress measurements using MitoSOX Red dye. CD4 T cells (FIGS. 6A-6B) and CD8 T cells (FIGS. 6C-6D) were transplanted with second organelle complexes (2nd OC) or vehicle and assessed at day 4.

FIGS. 7A-7D depict data related to cellular oxidative stress levels measured using Cell ROX Green dye. CD4 T cells (FIGS. 7A-7B) and CD8 T cells (FIGS. 7C-7D) were transplanted with second organelle complexes (2^{nd} OC) or vehicle and assessed at day 4.

FIG. 8 depicts data related to T cell oxygen consumption rate measured with a Seahorse Extracellular Flux Analyzer. T cells were transplanted with second organelle complexes (2^{nd} OC) or vehicle and assessed at day 4.

FIGS. 9A-9B depict data related to cytokine production capacity evaluated using PMA and Ionomycin. T cells were transplanted with second organelle complexes (2^{nd} OC) or vehicle and assessed at day 4.

FIGS. 10A-10B depict data related to the number of surviving cells after in vitro TCR chronic stimulation. T cells were transplanted with second organelle complexes (2^{nd} OC) or vehicle and assessment of viable cell count was performed using a dead cell staining dye at Day 9.

FIG. 11 depicts data related to CAR-T cell glycolytic rates measured with a Seahorse Extracellular Flux Analyzer.

FIGS. 12A-12D depict data related to CAR-T cell proliferative capacity evaluated by dye dilution assay during co-culture with A20. CD4 CAR-T cells (FIGS. 12A-12B) and CD8 CAR-T cells (FIGS. 12C-12D) were transplanted with second organelle complexes (2^{nd} OC) or vehicle.

FIGS. 13A-13B depict data related to cytokine production capacity evaluated using PMA and Ionomycin.

FIG. 14 depicts data related to a cytotoxicity assay performed by co-culturing with A20 cells.

FIG. 15 depicts data related to overall survival of mice injected with second organelle complexes-treated CAR-T cells as compared to vehicle-treated CAR-T cells or naïve T cells.

FIGS. 16A-16B depicts flow cytometric analysis data related to TCF-1 precursor cells. T cells were transplanted with second organelle complexes (2^{nd} OC) or vehicle and assessed after 72 hours of TCR stimulation.

FIGS. 17A-17B depicts flow cytometric analysis data related to TCF-1 precursor CAR-T cells. CAR-T cells were transplanted with second organelle complexes (2^{nd} OC) or vehicle and assessed 96 hours after the initiation of TCR stimulation.

FIGS. 18A-18B depict data related to GPX4 expression in CD4⁺ T cells (FIG. 18A) and CD8⁺ T cells (FIG. 18B) transplanted with second organelle complexes (Q) or vehicle after 72 hours stimulation with anti-CD3/CD28 antibodies.

FIGS. 19A-19B depict data related to lipid peroxidation levels in CD4⁺ T cells (FIG. 19A) and CD8⁺ T cells (FIG. 19B) transplanted with second organelle complexes (Q) or vehicle after 72 hours stimulation with anti-CD3/CD28 antibodies.

FIGS. 20A-20B depict data related to the percentage of apoptotic CD4⁺ T cells (FIG. 20A) and CD8⁺ T cells (FIG. 20B) transplanted with second organelle complexes (Q) or vehicle after 120 hours stimulation with anti-CD3/CD28 antibodies.

FIGS. 21A-21B depict data related to ROS accumulation of Vehicle CAR-T cells and Q-treated CAR-T cells assessed with CellROX Green dye in a CD4⁺ (FIG. 21A) and CD8⁺ (FIG. 21B) context.

FIGS. 22A-22B depict data related to lipid peroxidation levels in CD4⁺ CAR-T cells (FIG. 22A) and CD8⁺ CAR-T cells (FIG. 22B) transplanted with second organelle complexes (Q) or vehicle.

FIGS. 23A-23B depict data related to the percentage of CD4⁺ CAR-T cells (FIG. 23A) and CD8⁺ CAR-T cells (FIG. 23B) transplanted with second organelle complexes (Q) or vehicle in tumor infiltrating lymphocytes (TIL) at 17 days after tumor cell inoculation.

FIGS. 24A-24D depict data related to cellular ROS (FIGS. 24A-24B) and mitochondrial ROS (FIGS. 24C-24D) accumulation in CD4⁺ T cells (FIG. 24A, FIG. 24C) and CD8⁺ T cells (FIG. 24B, FIG. 24D) transplanted with second organelle complexes (Q) or vehicle after 192 hours stimulation with anti-CD3/CD28 antibodies.

FIGS. 25A-25B depict data related to the proliferation ability of CD4⁺ T cells (FIG. 25A) and CD8⁺ T cells (FIG. 25B) transplanted with second organelle complexes (Q) or vehicle after chronic stimulation.

FIGS. 26A-26B depict data related to viable cell percentage (FIG. 26A) and viable cell number (FIG. 26B) of T cells transplanted with second organelle complexes (Q) or vehicle after 192 hrs stimulation with anti-CD3/-CD28 antibodies.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, “isolated” shall be given its ordinary meaning and shall also refer to a substance or entity that has been (1) separated from at least some of the components with which it was associated when initially produced (whether in nature or in an experimental setting), and/or (2) produced, prepared, and/or manufactured by the hand of man. In some embodiments, an isolated mitochondrion or isolated organelle complexes population has been processed to obtain it from a cellular environment via the methods provided herein.

As used herein, the term “mitochondrion” shall be given its ordinary meaning and shall also refer to an organelle present in a eukaryotic cell that has double-layered lipid membranes, the inner and outer membranes, and a matrix surrounded by cristae and inner membranes. Mitochondria (more than one mitochondrion) have enzymes on their inner membrane, such as the respiratory chain complexes, which is involved in oxidative phosphorylation. The inner membrane has a membrane potential due to the internal-external proton gradients formed by the action of the respiratory chain complexes, etc. Mitochondria are thought to be unable to maintain the membrane potential when the inner membrane is disrupted.

As used herein, the term “organelle complex” shall be given its ordinary meaning and shall also refer to a complex of mitochondria and one or more of endoplasmic reticulum, peroxisomes, lysosomes, and Golgi apparatus. Organelle complexes can be depleted of cytosolic macromolecules (e.g., cytosolic proteins). In some embodiments, organelle complexes do not comprise cytosolic macromolecules. In some embodiments, an organelle complexes population comprises homogenized mitochondria. As used herein, the term “population” shall be given its ordinary meaning and shall also refer to a group of a plurality of the same or different substances. For example, an “organelle complexes population” is a group of at least a plurality of the same or different organelle complexes. The population may not be always homogenous and may have physical, chemical and/or physiological distributions. The physical distribution includes, for example, particle size and polydispersity index. The chemical distribution includes, for example, a zeta potential distribution and a lipid composition distribution. The physiological distribution includes, for example, a difference of physiological function (for example, respiratory activity). An organelle complexes population can comprise first organelle complexes, second organelle complexes, homogenized mitochondria, or any combination thereof. As used herein, the term “homogenized mitochondria” shall be given its ordinary meaning and shall also refer to mitochondria isolated via a method comprising one or more homogenization steps.

As used herein, the term “surfactant” shall be given its ordinary meaning and shall also refer to a molecule having a hydrophilic moiety and a hydrophobic moiety in one molecule. Surfactants have the role of reducing surface tension at the interface or mixing polar and non-polar substances by forming micelles. Surfactants are roughly classified into nonionic surfactants and ionic surfactants. Nonionic surfactants are those in which the hydrophilic moiety is not ionized, and ionic surfactants are those in which the hydrophilic moiety comprises either a cation or an anion or both a cation and an anion.

As used herein, the term “critical micelle concentration” (CMC) shall be given its ordinary meaning and shall also refer to the concentration at which, when the concentration is reached, the surfactant forms micelles, and the surfactant further added to the system contributes to micelle formation, in particular the concentration in bulk. At concentrations above the critical micelle concentration, the addition of surfactants to the system ideally increases the amount of micelles, especially the number of micelles.

As used herein, the term “modulation” shall be given its ordinary meaning and shall also refer to a change or an alteration in a biological activity. Modulation includes, but is not limited to, stimulating or inhibiting an activity. Modulation may be an increase or a decrease in activity, a change in binding characteristics, or any other change in the biological, functional, or immunological properties associated with the activity of a cell, a cell population, a protein, a pathway, a system, or other biological targets of interest, and can be a change of at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values).

As used herein, a “subject” refers to an animal that is the object of treatment, observation or experiment. “Animal” includes cold-and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. As used herein, the term “host cell” shall be given its ordinary meaning and shall also refer to an in vivo cell, an in vitro cell, and/or an ex vivo cell into which the incorporation of exogenous mitochondria and/or organelle complexes is intended.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “anti-tumor effects” shall be given its ordinary meaning and shall also refer to the inhibition of the formation, growth, and/or viability of tumors and/or tumor cells (in vivo and/or in vitro) and includes an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) change in one or more of tumor cell death, tumor volume, tumor number, tumor viability, metastasis, and survival rate.

As used herein, the term “oxidative stress” shall be given its ordinary meaning and shall also refer to an imbalance between generation of reactive oxygen species, reactive nitrogen species, and/or free radicals, and the antioxidative capacity of biological system.

As used herein, the term “effective amount” refers to an amount sufficient to effect beneficial or desirable biological and/or clinical results.

As used herein, the term “contacting” shall be given its ordinary meaning and shall also refer to placing two or more entities in such proximity such that they actually physically contact each other, e.g., by combining the two or more entities (e.g., isolated organelle complexes and T cells). Contacting can comprise co-incubation. Contacting can occur in vitro, in situ or in vivo. In some embodiments, contacting the two entities comprises incorporation (e.g., transplantation) of one entity into another entity physically contacted. Contacting isolated organelle complexes with a population of T cells can comprise contacting an organelle complexes population with a population of T cells. Contacting isolated organelle complexes with a population of T cells can generate a population of T cells comprising exogenous organelle complexes. Upon contact of the organelle complexes with a population of T cells, the organelle complexes provided herein can be capable of incorporating into the T cells. In some embodiments, incorporation of isolated organelle complexes (e.g., transplantation) into a T cell comprises colocalization and/or fusion with endogenous mitochondria within said T cell. The T cells can be in vivo, in vitro, or ex vivo. In some embodiments, organelle complexes that have been incorporated (e.g., transplanted) into T cells can be detected (e.g., distinguished from the endogenous organelles of the T cells) for at least a period of time (e.g., 6 hours, 12 hours, 16 hours, 1 day, 2 days, 3 days, 4 days, 5 days, 6 days, 7 days, or a number or a range between any two of these values). The beneficial effects of organelle complexes transplantation provided herein can persist beyond the time when said organelle complexes are detectable within a T cell population. T cell populations comprising organelle complexes provided herein include T cell populations that have undergone one or more cell divisions and/or continuous culture after initially having been contacted with the isolated organelle complexes. In some embodiments, the progeny of T cells that comprise organelle complexes also comprise said organelle complexes (e.g., via the random distribution of organelle complexes during division). As used herein, the term “exogenous” shall be given its ordinary meaning and shall also refer to cellular material (e.g., organelle complexes) that has removed from one cell and incorporated into another cell.

The term “autologous” shall be given its ordinary meaning, and shall also refer to any material derived from the same individual to whom it is later to be re-introduced into the individual.

The term “allogeneic” shall be given its ordinary meaning, and shall also refer to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The methods, compositions, systems, and kits provided herein can, in some embodiments, be employed in concert with the methods, compositions, systems, and kits described in PCT Patent Application Publication Nos. WO2018/092839, WO2017/090763, WO2020/230601, WO2019/164003, WO2020/054824, WO2020/203961, WO2020/054829, WO2021/015298, and WO2021/132735, the contents of which are incorporated herein by reference in their entirety.

There are provided, in some embodiments, methods of producing a population of T cells for adoptive T cell therapy. In some embodiments, the method comprises: contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus. The method can comprise stimulating the population of T cells. The stimulating step can expand the population of T cells.

There are provided, in some embodiments, methods of enhancing the proliferation, migration, persistence and/or activity of a population of T cells for adoptive T cell therapy. In some embodiments, the method comprises: contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus. The method can comprise: stimulating the population of T cells to expand the population of T cells.

There are provided, in some embodiments, methods of generating a population of T cells resistant to exhaustion. In some embodiments, the method comprises: contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus. The method can comprise: stimulating the population of T cells to expand the population of T cells.

In certain embodiments, the T cells are obtained from a donor subject. In some embodiments, the donor subject is human patient afflicted with a cancer or a tumor. In other embodiments, the donor subject is a human patient not afflicted with a cancer or a tumor.

The T cells provided herein may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain embodiments, the cells collected by apheresis are washed to remove the plasma fraction, and placed in an appropriate buffer or media for subsequent processing. In some embodiments, the cells are washed with PBS. As will be appreciated, a washing step can be used, such as by using a semiautomated flowthrough centrifuge, e.g., the Cobe™ 2991 cell processor, the Baxter CytoMate™, or the like. In some embodiments, the washed cells are resuspended in one or more biocompatible buffers, or other saline solution with or without buffer. In certain embodiments, the undesired components of the apheresis sample are removed. Additional methods of isolating T cells for a T cell therapy are disclosed in U.S. Patent Publication No. 2013/0287748, which is herein incorporated by references in its entirety.

In certain embodiments, T cells are isolated from PBMCs by lysing the red blood cells and depleting the monocytes, e.g., by using centrifugation through a PERCOLL™ gradient. In some embodiments, a specific subpopulation of T cells, such as CD4⁺, CD8⁺, CD28⁺, CD45RA⁺, and CD45RO⁺ T cells is further isolated by positive or negative selection techniques known in the art. For example, enrichment of a T cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4⁺ cells by negative selection, a monoclonal antibody cocktail typically includes antibodies to CD8, CD11b, CD14, CD16, CD20, and HLA-DR. In certain embodiments, flow cytometry and cell sorting are used to isolate cell populations of interest for use in the methods provided herein.

In some embodiments, the T cells are genetically modified following isolation using known methods, or the T cells are activated and expanded (or differentiated in the case of progenitors) in vitro prior to being genetically modified. In another embodiment, the T cells are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynaheads® system, a CD3/CD2S activator/stimulator system for physiological activation of human ‘I’ cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

The stimulating can be performed before the contacting, after the contacting, and/or during contacting. The term “stimulation,” shall be given its ordinary meaning, and shall also refer to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the signaling domains of the CAR. Stimulation can mediate altered expression of certain molecules. The stimulating can comprise culturing the population of T cells in the presence of one or more stimulating agents. The one or more stimulating agents can comprise an agent that stimulates a CD3/TCR complex-associated signal and an agent that stimulates a costimulatory molecule on the surface of the T cells. The one or more stimulating agents can comprise: a molecule that binds CD28 (e.g., one or more of an anti-CD28 antibody, CD80, and CD86); and/or a molecule that binds CD3 (e.g., an anti-CD3 antibody). The stimulating and/or contacting can be performed for a period time of at least about 6 hours, 12 hours, 16 hours, 1 day, 2 days, 3 days, 4 days, 5 days, 6 days, 7 days, or a number or a range between any two of these values.

In some embodiments, T cell recovery efficiency can be at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) greater as compared to a method that does not comprise contacting isolated organelle complexes with the population of T cells. T cell recovery efficiency can be the ratio of T cells recovered after stimulation (e.g., after about 48 hours of stimulation) to the number of T cells immediately before the start of stimulation. In some embodiments, the population of T cells exhibit one or more of enhanced expansion capability (in vivo and/or in vitro), enhanced cytotoxicity against target cells (in vivo and/or in vitro), enhanced resistance to exhaustion (in vivo and/or in vitro), and enhanced persistence (in vivo and/or in vitro), as compared to a population of T cells that do not comprise exogenous organelle complexes. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in basal and/or maximal oxygen consumption rate (in vivo and/or in vitro) as compared to a population of T cells that do not comprise exogenous organelle complexes. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in basal and/or maximal glycolytic rate (in vivo and/or in vitro) as compared to a population of T cells that do not comprise exogenous organelle complexes. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in glycolytic capacity and/or respiratory capacity (in vivo and/or in vitro) as compared to a population of T cells that do not comprise exogenous organelle complexes. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in mitochondrial mass (in vivo and/or in vitro) as compared to a population of T cells that do not comprise exogenous organelle complexes. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in cytotoxic activity against target cells (in vivo and/or in vitro) as compared to a population of T cells that do not comprise exogenous organelle complexes. The exhaustion markers can be selected from the group comprising PD-1, CTLA-4, TIM-3, LAG-3, BTLA, 2B4, CD 160, CD39, VISTA, TIGIT, or any combination thereof. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) reduction in levels one or more of cellular ROS (in vivo and/or in vitro), mitochondrial ROS (in vivo and/or in vitro), cellular oxidative stress (in vivo and/or in vitro), and mitochondrial oxidative stress (in vivo and/or in vitro), as compared to a population of T cells that do not comprise exogenous organelle complexes. The reactive oxygen species can comprise superoxide (O₂·⁻), hydroperoxy (HO·₂), hydrogen peroxide (H₂O₂), peroxynitrite (ONOO⁻), hypochlorous acid (HOCl), hypobromous acid (HOBr), hydroxyl radical (HO·), peroxy radical (ROO·), alkoxy radical (RO·), singlet oxygen (¹O₂), lipid peroxides, lipid peroxyradicals or lipid alkoxyl radicals, or any combination thereof. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase production of one or more of cytokines (in vivo and/or in vitro) as compared to a population of T cells that do not comprise exogenous organelle complexes. The cytokines can comprise interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in cell proliferation (in vivo and/or in vitro) as compared to a population of T cells that do not comprise exogenous organelle complexes. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in cell viability following chronic TCR stimulation (in vivo and/or in vitro) as compared to a population of T cells that do not comprise exogenous organelle complexes. In some embodiments, the introduction of organelle complexes metabolically reprograms the CAR-T cells via redox modulation. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) decrease and/or increase in one or more of NAD⁺/NADH, ATP levels, Sirtuin (Sirt1,3) levels, TP-53 levels, NF-κB levels, Bax levels, memory T cell (Tcm, Tscm) percentage, telomere activity, and SA-β Gal activity (in vivo and/or in vitro) as compared to a population of T cells that do not comprise exogenous organelle complexes.

“Exhaustion” or “unresponsiveness” refers to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation or cell division, entrance into the cell cycle, cytokine production, cytotoxicity, trafficking, phagocytotic activity, or any combination thereof. Normal input signals can include, but are not limited to, stimulation via a receptor (e.g., T cell receptor, B cell receptor, co-stimulatory receptor, and the like).

Exhausted immune cells can have a reduction of at least 10%, 15%, 20%, 25%, 30%, 35%, 40%, 45%, 50%, 55%, 60%, 65%, 70%, 75%, 80%, 85%, 90%, 95%, 99% or more in cytotoxic activity, cytokine production, proliferation, trafficking, phagocytotic activity, or any combination thereof, relative to a corresponding control immune cell of the same type. In one embodiment, a cell that is exhausted is a CD8⁺ T cell (e.g., an effector CD8⁺ T cell that is antigen-specific). CD8 cells normally proliferate (e.g., clonally expand) in response to T cell receptor and/or co-stimulatory receptor stimulation, as well as in response to cytokines such as IL-2. Thus, an exhausted CD8 T cell is one which does not proliferate and/or produce cytokines in response to normal input signals. It is well known that the exhaustion of effector functions can be delineated according to several stages, which eventually lead to terminal or full exhaustion and, ultimately, deletion (Yi et al. (2010) Immunol. 129:474-481; Wherry and Ahmed (2004) J. Virol. 78:5535-5545). In the first stage, functional T cells enter a “partial exhaustion I” phase characterized by the loss of a subset of effector functions, including loss of IL-2 production, reduced TNFa production, and reduced capacity for proliferation and/or ex vivo lysis ability. In the second stage, partially exhausted T cells enter a “partial exhaustion II” phase when both IL-2 and TNFa production ceases following antigenic stimulation and IFNy production is reduced. “Full exhaustion” or “terminal exhaustion” occurs when CD8⁺ T cells lose all effector functions, including the lack of production of IL-2, TNFa, and IFNy and loss of ex vivo lytic ability and proliferative potential, following antigenic stimulation. A fully exhausted CD8⁺ T cell is one which does not proliferate, does not lyse target cells (cytotoxicity), and/or does not produce appropriate cytokines, such as IL-2, TNFa, or IFNy, in response to normal input signals. Such lack of effector functions can occur when the antigen load is high and/or CD4 help is low. This hierarchical loss of function is also associated with the expression of co-inhibitor immune receptors, such as PD-1, ‘HM-3, LAG-3, and the like (Day et al. (2006) Nature 443:350-4; Trauttnann et al. (2006) Nat. Med 12: 1198-202; and Urbani et al. (2006) J. Virol. 80: 1398-1403). Other molecular markers distinguish the hierarchical stages of immune cell exhaustion, such as high eomesodermin (EOMES) and low TBET expression as a marker of terminally exhausted T cells (Paley et al. (2012) Science 338: 1220-1225). Additional markers of exhausted T cells, such as the reduction of Bcl-b and the increased production of BLIMP-1 (Pdrm1). T cell exhaustion can comprise expression of one or more T cell exhaustion biomarkers selected from the group comprising a checkpoint inhibitor, PD-1 (Pdcd1), TπM-3 (Havcr2), LAG-3 (Lag3), CTLA-4 (Ctla4), 2B4 (CD244), CD39 (Entpd1), CD 160, eomesodermin (Eomes), T-BET (Tbx21), BATF, BLIMP-1 (Prdm1), NFATC1, NR4A2, MAFB, OCT-2 (Pou2f2), Foxpl, retinoic acid receptor alpha (Rara), or any combination thereof. In some embodiments, the population of T cells exhibit an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) decrease in the level of one or more exhaustion markers (in vivo and/or in vitro) as compared to a population of T cells that do not comprise exogenous organelle complexes. The in vivo persistence of the population of T cells can comprise a period of about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, or a number or a range between any of these values.

There are provided, in some embodiments, populations of T cells generated by a method disclosed herein. There are provided, in some embodiments, populations of T cells for an adoptive T cell therapy wherein the T cells comprise exogenous organelle complexes. The organelle complexes can comprise mitochondria and one or more of endoplasmic reticulum, peroxisomes, lysosomes, and Golgi apparatus. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

There are provided, in some embodiments, methods of treating or preventing a disease or disorder in a subject. In some embodiments, the method comprises: administering to the subject an effective amount of the population of T cells generated according to a method disclosed herein, thereby treating or preventing the disease or disorder in the subject. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

There are provided, in some embodiments, methods for enhancing adoptive T cell therapy in a subject. In some embodiments, the method comprises: generating an effective amount of T cells comprising exogenous organelle complexes according to a method disclosed herein; and adoptively transferring said T cells to the subject. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

There are provided, in some embodiments, methods of treating or preventing a disease or disorder in a subject. In some embodiments, the method comprises: administering to the subject an effective amount of a population of T cells (e.g., T cells comprising a chimeric antigen receptor (CAR) and/or an engineered T cell receptor (TCR)); and administering to the subject an effective amount of isolated organelle complexes, thereby treating or preventing the disease or disorder in the subject. The isolated organelle complexes can be capable of contacting the population of T cells in vivo to generate a population of T cells comprising the organelle complexes. In some embodiments, the population of T cells exhibit one or more of enhanced expansion capability, enhanced cytotoxicity against target cells, enhanced resistance to exhaustion, and enhanced persistence, as compared to a population of T cells that do not comprise exogenous organelle complexes. The subject can be administered an effective amount of the population of T cells prior to administering to the subject an effective amount of isolated organelle complexes. The subject can be administered an effective amount of isolated organelle complexes prior to administering to the subject an effective amount of the population of T cells. The subject can be administered an effective amount of the population of T cells and an effective amount of isolated organelle complexes simultaneously.

Disclosed herein include methods of treating or preventing a disease or disorder in a subject. In some embodiments, the method comprises: administering to the subject an effective amount of a heterologous nucleic acid (e.g., a vector, a viral vector) encoding a chimeric antigen receptor (CAR) and/or an engineered T cell receptor (TCR); and administering to the subject an effective amount of isolated organelle complexes, thereby treating or preventing the disease or disorder in the subject. The heterologous nucleic acid and isolated organelle complexes can be capable of contacting T cells of the subject in vivo to generate T cells comprising the organelle complexes and a CAR and/or an engineered TCR. In some embodiments, said T cells exhibit one or more of enhanced expansion capability, enhanced cytotoxicity against target cells, enhanced resistance to exhaustion, and enhanced persistence, as compared to T cells that do not comprise exogenous organelle complexes.

In some embodiments, the population of T cells reduces tumor volume, tumor growth, and/or tumor burden in the subject. In some embodiments, the population of T cells reduces tumor volume in the subject at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared with the tumor volume in an untreated subject or a subject administered a population of T cells that do not comprise exogenous organelle complexes. In some embodiments, the population of T cells increases overall survival or progression-free survival. In some embodiments, the population of T cells increases overall survival or progression-free survival at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared with untreated subjects or subjects administered a population of T cells that do not comprise exogenous organelle complexes.

The T cells can be autologous to the subject. The T cells can be allogenic to the subject. The adoptive T cell therapy can be a CAR-T cell therapy. The adoptive T cell therapy can be an engineered TCR T cell therapy. The adoptive T cell therapy can be a tumor infiltrating lymphocyte (TIL) therapy. The administering can comprise administering (e.g., intravenously) at least about 1×10⁶ T cells. The method can comprise repeated administrations of the population of T cells. The subject can be a mammal.

The disease or disorder can be associated with expression of a tumor antigen. The disease associated with expression of a tumor antigen can be selected from the group consisting of a proliferative disease, a precancerous condition, a cancer, and a non-cancer related indication associated with expression of the tumor antigen.

The cancer can be selected from the group consisting of colon cancer, rectal cancer, renal-cell carcinoma, liver cancer, small cell or non-small cell carcinoma of the lung, mesothelioma, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers, combinations of said cancers, and metastatic lesions of said cancers.

The cancer can be a hematologic cancer chosen from one or more of chronic lymphocytic leukemia (CLL), acute leukemias, acute lymphoid leukemia (ALL), B-cell acute lymphoid leukemia (B-ALL), T cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell-or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

### Organelle Complexes

In some embodiments, upon contact of the isolated organelle complexes with a population of T cells, the organelle complexes are capable of incorporating into the T cells. The contacting step can be repeated at least 2, 3, or 4 times. The contacting can be performed during one or both of the stimulating step and the introducing step. The population of T cells can be contacted with an effective amount of isolated organelle complexes sufficient to enhance the proliferation, migration, persistence and/or activity of said T cells. The effective amount can comprise about 20 ug of isolated organelle complexes. The effective amount of isolated organelle complexes can be, can be about, can be at least, or can be at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, ug, mg, ug/mL, and/or mg/mL. The population of T cells can be contacted with an effective amount of isolated organelle complexes sufficient to render the said T cells resistant to exhaustion. The population of T cells can be derived from lymph node cells (e.g., purified by a magnetic particle-based enrichment procedure selected from the group consisting of manual MACS®, AutoMACS®, CliniMACS®, EasySep®, and RoboSep®). The population of T cells can comprise one or more of a CD4⁺ cell, a CD8⁺ T cell, a cytotoxic T cell, a terminal effector T cell, an effector T cell, a memory or central memory T cell, a naive T cell, a regulatory T cell, a natural killer T cell, a gamma-delta T cell, a cytokine induced killer (CIK) T cell and a tumor infiltrating lymphocyte (TIL).

The organelle complexes provided herein can comprise first organelle complexes, second organelle complexes, or a combination of first organelle complexes and second organelle complexes. Isolated organelle complexes can be an organelle complexes population. The organelle complexes population can comprise first organelle complexes, or a combination of first organelle complexes and second organelle complexes. The first organelle complexes and/or second organelle complexes can be depleted of cytosolic macromolecules. The cytosolic macromolecules can comprise cytosolic proteins (e.g., the cytosolic proteins are p70S6K and/or glyceraldehyde 3-phosphate dehydrogenase (GAPDH)). The abundance of one or more cytosolic proteins can be depleted by at least about 90% (e.g., 90%, 91%, 92%, 93%, 94%, 95%, 96%, 97%, 98%, 99%, 100%, or a number or a range between any two of these values) as compared to the cells from which the organelle complexes population are derived. The organelle complexes can comprise: one or more mitochondrial matrix proteins (e.g., mitochondrial transcription factor A (TFAM) and/or citrate synthase (CS)); one or more outer mitochondrial membrane proteins (e.g., outer mitochondrial membrane complex subunit 20 (TOMM20)); one or more lysosome proteins (e.g., lysosomal-associated membrane protein 2 (LAMP2), mannose-6-phosphate receptor (M6PR), and/or lysosomal-associated membrane protein 1 (LAMP1)); one or more peroxisome proteins (e.g., catalase and/or ATP-binding cassette transporter 1, subfamily D, type 3 (ABCD3)); one or more Golgi apparatus proteins (e.g., Golgin-97, Sintaxin-6, TGOLN2/trans-Golgi network protein 2 (TGN46), Golgi matrix protein 130 (GM130), and/or Mannosidase Alpha Class 2A Member 1 (MAN2A1)); and/or one or more endoplasmic reticulum proteins (e.g., Calreticulin and/or Calnexin). The organelle complexes can be derived from cells of a subject different from the subject from which the T cells are derived. The organelle complexes can be derived from cells of the same subject from which the T cells are derived. The organelle complexes (e.g., first organelle complexes, second organelle complexes) provided herein can comprise mitochondria and one, two, three, or four of endoplasmic reticulum, peroxisomes, lysosomes, and Golgi apparatus. The organelle complexes (e.g., first organelle complexes, second organelle complexes) can comprise: (i) mitochondria and endoplasmic reticulum; (ii) mitochondria and peroxisomes; (iii) mitochondria and lysosomes; (iv) mitochondria and Golgi apparatus; (v) mitochondria, endoplasmic reticulum, and peroxisomes; (vi) mitochondria, endoplasmic reticulum, and lysosomes; (vii) mitochondria, endoplasmic reticulum, and Golgi apparatus; (viii) mitochondria, endoplasmic reticulum, peroxisomes, and lysosomes; (ix) mitochondria, endoplasmic reticulum, peroxisomes, and Golgi apparatus; (x) mitochondria, endoplasmic reticulum, peroxisomes, lysosomes, and Golgi apparatus; (xi) mitochondria, endoplasmic reticulum, lysosomes, and Golgi apparatus; (xii) mitochondria, peroxisomes, and lysosomes; (xiii) mitochondria, peroxisomes, and Golgi apparatus; (xiv) mitochondria, peroxisomes, lysosomes, and Golgi apparatus; and/or (xv) mitochondria, lysosomes, and Golgi apparatus. The ratio of mitochondria to additional organelles (e.g., endoplasmic reticulum, peroxisomes, lysosomes, and/or Golgi apparatus) in the organelle complexes population can vary.

Disclosed herein include methods for generating first organelle complexes populations. In some embodiments, the method comprises: incubating cells in a first solution comprising a surfactant at a first temperature; removing the surfactant to form a second solution; and recovering first organelle complexes from the second solution. First organelle complexes can be derived from: (i) frozen cells; (ii) floating cells; and/or (iii) cells contacted with a surfactant at a concentration at or above the critical micellar concentration (CMC) for the surfactant. The generation of first organelle complexes can comprise: (Step A) providing adherent, floating, and/or frozen cells, thawing, and placing in a tube. The generation of first organelle complexes can comprise: (Step A) providing adherent, floating, and/or frozen cells, centrifuging, and collecting the precipitant. The generation of first organelle complexes can comprise: (Step A) providing adherent cells, aspirating, adding a solution (e.g., PBS(−)), aspirating, adding TrypLE, incubating, adding a solution (e.g., PBS(−)), placing the cell suspension in a tube, centrifuging, and collecting the precipitant. The generation of first organelle complexes can comprise one or more of the following steps: (Step B) adding Tris Buffer, centrifuging, and collecting the precipitant; (Step C) adding Tris Buffer and vortexing; (Step D) adding a solution comprising a surfactant and incubating; (Step E) centrifuging and collecting the precipitant; (Step F) adding Tris Buffer, centrifuging and collecting the precipitant; (Step G) adding Tris Buffer and pipetting; (Step H) transferring to another tube and collecting buffer solution in the original tube and rinsing it; (Step I) centrifuging and collecting the supernatant; (Step J) centrifuging and collecting the precipitant; and (Step K) pipetting. One or more of the above steps can comprise an incubation period. One or more of the above steps can comprise a centrifugation step, followed by collection of the supernatant and/or the precipitant. One or more of the above steps can be omitted and one or more additional steps can be included. The times, volumes, concentrations, and centrifugal forces can vary depending on the embodiment. Incubating cells in the first solution and/or incubating the second solution can comprise applying a physical stimulus to the first solution and/or the second solution, respectively, such as, for example, pipetting, shaking and/or stirring. Applying a physical stimulus to the first solution and/or the second solution can comprise flowing the first solution and/or the second solution through a flow device (e.g., a reducer flow device). Said flow device can comprise a fluidic channel comprising two or more segments of varying cross-sectional diameters. Said cross-sectional diameters can be, can be about, can be at least, or can be at most, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or a number or a range between any two of these values. Reducer flow devices provided herein can have various configurations, such as, for example, square reducers, tapered reducers, concentric reducers, and/or eccentric reducers. The flow device can comprise various types and sizes of tubes to create additional flow and shear for first organelle complexes extraction. In some embodiments, said flowing through the flow device generates additional flow and/or shear. Recovering the first organelle complexes from the second solution can comprise tangential flow filtration (TFF). TFF can be employed for purification and/or buffer exchange in some embodiments. Recovering the first organelle complexes from the second solution can comprise tangential flow filtration (TFF). TFF can be performed with a low viscosity buffer, and in some embodiments said low viscosity buffer reduces the shear rate. The viscosity of the TFF buffer can be, can be about, can be at least, or can be at most, 1 centipoise (cP), 2 cP, 3 cP, 4 cP, 5 cP, 6 cP, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, 50 cP, 51 cP, 52 cP, 53 cP, 54 CP, 55 cP, 56 cP, 57 cP, 58 cP, 59 cP, 60 cP, 61 cP, 62 cP, 63 cP, 64 cP, 65 cP, 66 cP, 67 cP, 68 cP, 69 cP, 70 cP, 71 cP, 72 cP, 73 cP, 74 cP, 75 cP, 76 cP, 77 cP, 78 cP, 79 cP, 80 cP, 81 cP, 82 cP, 83 cP, 84 cP, 85 cP, 86 cP, 87 cP, 88 cP, 89 cP, 90 cP, 91 cP, 92 cP, 93 cP, 94 cP, 95 cP, 96 cP, 97 cP, 98 cP, 99 cP, 100 cP, 200 cP, 300 cP, 400 cP, 500 cP, 600 cP, 700 cP, 800 cP, 900 cP, 1000 cP, 2500 cP, 5000 cP, 7500 cP, 10000 cP, or a number or a range between any two of these values. The temperature at which TFF is performed can be, can be about, can be at least, or can be at most, 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or a number or a range between any two of these values. The shear rate of the TFF procedure and/or flow device (e.g., reducer flow device) can be, can be about, can be at least, or can be at most, 1 sec⁻¹, 2 sec⁻¹, 3 sec⁻¹, 4 sec⁻¹, 5 sec⁻¹, 6 sec⁻¹, 7 sec⁻¹, 8 sec⁻¹, 9 sec⁻¹, 10 sec⁻¹, 11 sec⁻¹, 12 sec⁻¹, 13 sec⁻¹, 14 sec⁻¹, 15 sec⁻¹, 16 sec⁻¹, 17 sec⁻¹, 18 sec⁻¹, 19 sec⁻¹, 20 sec⁻¹, 25 sec⁻¹, 30 sec⁻¹, 35 sec⁻¹, 40 sec⁻¹, 45 sec⁻¹, 50 sec⁻¹, 60 sec⁻¹, 70 sec⁻¹, 80 sec⁻¹, 90 sec⁻¹, 100 sec⁻¹, 110 sec⁻¹, 120 sec⁻¹, 128 sec⁻¹, 130 sec⁻¹, 140 sec⁻¹, 150 sec⁻¹, 160 sec⁻¹, 170 sec⁻¹, 180 sec⁻¹, 190 sec⁻¹, 200 sec⁻¹, 210 sec⁻¹, 220 sec⁻¹, 230 sec⁻¹, 240 sec⁻¹, 250 sec⁻¹, 260 sec⁻¹, 270 sec⁻¹, 280 sec⁻¹, 290 sec⁻¹, 300 sec⁻¹, 310 sec⁻¹, 320 sec⁻¹, 330 sec⁻¹, 340 sec⁻¹, 350 sec⁻¹, 360 sec⁻¹, 370 sec⁻¹, 380 sec⁻¹, 390 sec⁻¹, 400 sec⁻¹, 410 sec⁻¹, 420 sec⁻¹, 430 sec⁻¹, 440 sec⁻¹, 450 sec⁻¹, 460 sec⁻¹, 470 sec⁻¹, 480 sec⁻¹, 490 sec⁻¹, 500 sec⁻¹, 510 sec⁻¹, 520 sec⁻¹, 530 sec⁻¹, 540 sec⁻¹, 550 sec⁻¹, 560 sec⁻¹, 570 sec⁻¹, 580 sec⁻¹, 590 sec⁻¹, 600 sec⁻¹, 610 sec⁻¹, 620 sec⁻¹, 630 sec⁻¹, 640 sec⁻¹, 650 sec⁻¹, 660 sec⁻¹, 670 sec⁻¹, 680 sec⁻¹, 690 sec⁻¹, 700 sec⁻¹, 710 sec⁻¹, 720 sec⁻¹, 730 sec⁻¹, 740 sec⁻¹, 750 sec⁻¹, 760 sec⁻¹, 770 sec⁻¹, 780 sec⁻¹, 790 sec⁻¹, 800 sec⁻¹, 810 sec⁻¹, 820 sec⁻¹, 830 sec⁻¹, 840 sec⁻¹, 850 sec⁻¹, 860 sec⁻¹, 870 sec⁻¹, 880 sec⁻¹, 890 sec⁻¹, 900 sec⁻¹, 910 sec⁻¹, 920 sec⁻¹, 930 sec⁻¹, 940 sec⁻¹, 950 sec⁻¹, 960 sec⁻¹, 970 sec⁻¹, 980 sec⁻¹, 990 sec⁻¹, 1000 sec⁻¹, 1100 sec⁻¹, 1200 sec⁻¹, 1300 sec⁻¹, 1400 sec⁻¹, 1500 sec⁻¹, 1600 sec⁻¹, 1700 sec⁻¹, 1800 sec⁻¹, 1900 sec⁻¹, 2000 sec⁻¹, 2100 sec⁻¹, 2200 sec⁻¹, 2300 sec⁻¹, 2400 sec⁻¹, 2500 sec⁻¹, 2600 sec⁻¹, 2700 sec⁻¹, 2800 sec⁻¹, 2900 sec⁻¹, 3000 sec⁻¹, 3250 sec⁻¹, 3500 sec⁻¹, 3750 sec⁻¹, 4000 sec⁻¹, 4250 sec⁻¹, 4500 sec⁻¹, 4750 sec⁻¹, 5000 sec⁻¹, 5500 sec⁻¹, 6000 sec⁻¹, 6500 sec⁻¹, 7000 sec⁻¹, 7500 sec⁻¹, 8000 sec⁻¹, 8500 sec⁻¹, 9000 sec⁻¹, 9500 sec⁻¹, 10000 sec⁻¹, or a number or a range between any two of these values. TFF can be performed with a buffer comprising human albumin (HA). Recovering the first organelle complexes from the second solution can comprise TFF performed using a TFF membrane. The molecular weight cutoff of the TFF membrane can be, can be about, can be at least, or can be at most, 10 kDa, 11 kDa, 12 kDa, 13 kDa, 14 kDa, 15 kDa, 16 kDa, 17 kDa, 18 kDa, 19 kDa, 20 kDa, 25 kDa, 30 kDa, 35 kDa, 40 kDa, 45 kDa, 50 kDa, 60 kDa, 70 kDa, 80 kDa, 90 kDa, 100 kDa, 110 kDa, 120 kDa, 128 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 410 kDa, 420 kDa, 430 kDa, 440 kDa, 450 kDa, 460 kDa, 470 kDa, 480 kDa, 490 kDa, 500 kDa, 510 kDa, 520 kDa, 530 kDa, 540 kDa, 550 kDa, 560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, 710 kDa, 720 kDa, 730 kDa, 740 kDa, 750 kDa, 760 kDa, 770 kDa, 780 kDa, 790 kDa, 800 kDa, 810 kDa, 820 kDa, 830 kDa, 840 kDa, 850 kDa, 860 kDa, 870 kDa, 880 kDa, 890 kDa, 900 kDa, 910 kDa, 920 kDa, 930 kDa, 940 kDa, 950 kDa, 960 kDa, 970 kDa, 980 kDa, 990 kDa, 1000 kDa, 1100 kDa, 1200 kDa, 1300 kDa, 1400 kDa, 1500 kDa, 1600 kDa, 1700 kDa, 1800 kDa, 1900 kDa, 2000 kDa, 2100 kDa, 2200 kDa, 2300 kDa, 2400 kDa, 2500 kDa, 2600 kDa, 2700 kDa, 2800 kDa, 2900 kDa, 3000 kDa, 3250 kDa, 3500 kDa, 3750 kDa, 4000 kDa, 4250 kDa, 4500 kDa, 4750 kDa, 5000 kDa, 5500 kDa, 6000 kDa, 6500 kDa, 7000 kDa, 7500 kDa, 8000 kDa, 8500 kDa, 9000 kDa, 9500 kDa, 10000 kDa, or a number or a range between any two of these values. Methods of obtaining organelle complexes from cells and organelle complexes obtained by such methods are disclosed in PCT Patent Application No. PCT/US23/27014, entitled “ORGANELLE COMPLEXES,” filed Jul. 6, 2023, the contents of which are incorporated herein by reference in its entirety. There are provided, in some embodiments, second organelle complexes. In some embodiments, the method for isolating second organelle complexes from cells comprises treating cells in a first solution with a surfactant at a concentration below the critical micelle concentration (CMC) for the surfactant, removing the surfactant to form a second solution, incubating the cells in the second solution, and recovering second organelle complexes from the second solution. Second organelle complexes can be derived from: (i) adherent cells; and/or (ii) cells contacted with a surfactant at a concentration below the critical micellar concentration (CMC) for the surfactant. There are provided in some embodiments, Q mitochondria. Second organelle complexes can comprise or be Q mitochondria. Methods of obtaining Q mitochondria from cells and Q mitochondria obtained by such methods are disclosed in PCT Patent Application Publication No. WO/2021/015298, the contents of which are incorporated herein by reference in its entirety. The organelle complexes population can be derived from cells treated with a mitochondria-activating agent (e.g., resveratrol). The organelle complexes can be depleted of cytosolic macromolecules. Cytosolic macromolecules can be absent from the organelle complexes populations provided herein. Organelle complexes (e.g., first organelle complexes, second organelle complexes) populations provided herein can comprise a negligible and/or undetectable amount of cytosolic macromolecules. The cytosolic macromolecules can comprise cytosolic proteins (e.g., p70S6K and/or glyceraldehyde 3-phosphate dehydrogenase (GAPDH)). The first organelle complexes and second organelle complexes can be derived from cells treated with a mitochondria-activating agent. The homogenized mitochondria, first organelle complexes, and/or second organelle complexes can be encapsulated in lipid membrane-based vesicles. Methods of encapsulating in lipid membrane-based vesicles are disclosed in PCT Patent Application Publication No. WO2021/132735, the contents of which are incorporated herein by reference in its entirety.

### Chimeric Antigen Receptors and Engineered T Cell Receptors

The T cells provided herein can comprise a chimeric antigen receptor (CAR) or T cell receptor (TCR). In some embodiments, the CAR comprises a T cell receptor (TCR) antigen binding domain. The method can comprise introducing a heterologous nucleic acid (e.g., a vector) encoding a chimeric antigen receptor (CAR) and/or an engineered T cell receptor (TCR) into the T cells. The introducing step can be performed before the contacting, after the contacting, and/or during the contacting. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some embodiments, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR-T, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9(CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in a CAR provided herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof.

In some embodiments, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR provided herein comprises an antibody fragment. In a further aspect, the CAR comprises an antibody fragment that comprises a scFv.

In some embodiments, the CAR provided herein comprises a target-specific binding element otherwise referred to as an antigen binding domain. The choice of moiety depends upon the type and number of ligands that define the surface of a target cell.

In some embodiments, the CAR-mediated T cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising anticomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs provided herein include CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8⁺ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The tumor antigen can be a solid tumor antigen. The tumor antigen can be selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Ab1) (bcr-ab1); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1 B1 (CYP1B1); CCCTC-Binding Factor (Zinc Finger Protein)-Like (BORIS or Brother of the Regulator of Imprinted Sites), Squamous Cell Carcinoma Antigen Recognized By T cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2 DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRTAM, Ly9(CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Pharmaceutically Acceptable Compositions and Methods of Administration

There are provided, in some embodiments, pharmaceutical compositions. In some embodiments, the pharmaceutical composition comprises: a population of T cells generated by a method disclosed herein; and one or more pharmaceutically acceptable carriers.

The present disclosure also provides use of a population of T cells comprising the organelle complexes in the manufacture of a medicament for treating the diseases and disorders provided herein. In some embodiments, the population of T cells comprising the organelle complexes is administered to the subject in combination with one or more additional agents and/or additional therapies designed to treat the disease or disorder.

Contacting cells of the subject can comprise a route of administration selected from the group comprising intravenous administration, intra-arterial administration, intra-tracheal administration, subcutaneous administration, intramuscular administration, inhalation, intrapulmonary administration, and intra-ocular administration. The population of T cells comprising the organelle complexes can be administered locally or systemically.

The wording “local administration” or “topic administration” as used herein indicates any route of administration by which a population of T cells comprising the organelle complexes is brought in contact with the body of the individual, so that the resulting T cells location in the body is topic (limited to a specific tissue, organ or other body part where the imaging is desired). Exemplary local administration routes include injection into a particular tissue by a needle, gavage into the gastrointestinal tract, and spreading a solution containing a population of T cells comprising the organelle complexes on a skin surface.

The wording “systemic administration” as used herein indicates any route of administration by which a population of T cells comprising the organelle complexes is brought in contact with the body of the individual, so that the resulting T cells location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In another aspect, this disclosure provides pharmaceutically acceptable compositions which comprise a therapeutically-effective amount of a population of T cells comprising the organelle complexes disclosed herein. As described in detail below, the pharmaceutical compositions of this disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the population of T cells comprising the organelle complexes. The pharmaceutical compositions can comprise one or more pharmaceutically-acceptable carriers. The phrase “therapeutically-effective amount” as used herein can refer to that amount of a population of T cells comprising the organelle complexes disclosed herein which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, vehicle, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of this disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., a population of T cells comprising the organelle complexes) which can be combined with a carrier material to produce a single dosage form will vary depending upon the host being treated, the particular mode of administration. The amount of active ingredient, which can be combined with a carrier material to produce a single dosage form will generally be that amount of the population of T cells comprising the organelle complexes which produces a therapeutic effect. Generally, out of one hundred percent, this amount will range from about 1% to about 99% of active ingredient, preferably from about 5% to about 70%, most preferably from about 10% to about 30%.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of T cells comprising the organelle complexes include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions and the like, are also contemplated as being within the scope of this disclosure.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Actual dosage levels of the active ingredients in the pharmaceutical compositions of this disclosure may be determined by the methods of this disclosure so as to obtain an amount of the active ingredient, which is effective to achieve the desired therapeutic response for a particular subject, composition, and mode of administration, without being toxic to the subject.

The T cells comprising the organelle complexes can be administered at a therapeutically effective amount. For example, a therapeutically effective amount of the T cells comprising the organelle complexes can be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells comprising the organelle complexes is about 10⁴ cells, about 10⁵ cells, about 10⁶ cells, about 10⁷ cells, or about 10⁸ cells. In one particular embodiment, the therapeutically effective amount of the T cells comprising the organelle complexes is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

Also provided herein are kits comprising one or more compositions (e.g., a formulation comprising a population of T cells comprising the organelle complexes) described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

### Additional Agents

In some embodiments, the method comprises administering one or more additional agents to the subject. In some embodiments, the one or more additional agents increases the efficacy of the T cells comprising exogenous organelle complexes. The one or more additional agents can comprise a protein phosphatase inhibitor, a kinase inhibitor, a cytokine, an inhibitor of an immune inhibitory molecule, and/or or an agent that decreases the level or activity of a TREG cell. The one or more additional agents can comprise an immune modulator, an anti-metastatic, a chemotherapeutic, a hormone or a growth factor antagonist, an alkylating agent, a TLR agonist, a cytokine antagonist, a cytokine antagonist, or any combination thereof. The one or more additional agents can comprise an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

The one or more additional agents can be selected from the group consisting of alkylating agents (nitrogen mustards, ethylenimine derivatives, alkyl sulfonates, nitrosoureas and triazenes); uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®); bendamustine (Treakisym®, Ribomustin®, Treanda®); chlormethine (Mustargen®); cyclophosphamide (Cytoxan®, Neosar®, Clafen®, Endoxan®, Procytox®, Revimmune™); ifosfamide (Mitoxana®); melphalan (Alkeran®); Chlorambucil (Leukeran®); pipobroman (Amedel®, Vercyte®); triethylenemelamine (Hemel®, Hexylen®, Hexastat®); triethylenethiophosphoramine; Temozolomide (Temodar®); thiotepa (Thioplex®); busulfan (Busilvex®, Myleran®); carmustine (BiCNU®); lomustine (CeeNU®); streptozocin (Zanosar®); estramustine (Emcyt®, Estracit®); fotemustine; irofulven; mannosulfan; mitobronitol; nimustine; procarbazine; ranimustine; semustine; triaziquone; treosulfan; and Dacarbazine (DTIC-Dome®); anti-EGFR antibodies (e.g., cetuximab (Erbitux®), panitumumab (Vectibix®), and gefitinib (Iressa®)); anti-Her-2 antibodies (e.g., trastuzumab (Herceptin®) and other antibodies from Genentech); antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), carmofur, cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®), decitabine (Dacogen®), enocitabine (Sunrabin®), sapacitabine, tegafur-uracil, tiazofurine, tioguanine, trofosfamide, and gemcitabine (Gemzar®); vinca alkaloids: vinblastine (Velban®), Velsar®), vincristine (Vincasar®, Oncovin®), vindesine (Eldisine®), vinorelbine (Navelbine®), vinflunine (Javlor®); platinum-based agents: carboplatin (Paraplat®, Paraplatin®), cisplatin (Platinol®), oxaliplatin (Eloxatin®), nedaplatin, satraplatin, and triplatin; anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®), aclarubicin, amrubicin, liposomal doxorubicin, liposomal daunorubicin, pirarubicin, pixantrone, and zorubicin; topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101), belotecan, and rubitecan; taxanes: paclitaxel (Taxol®), docetaxel (Taxotere®), larotaxel, cabazitaxel, ortataxel, and tesetaxel; antibiotics: actinomycin (Cosmegen®), bleomycin (Blenoxane®), hydroxyurea (Droxia®, Hydrea®), mitomycin (Mitozytrex®, Mutamycin®); immunomodulators: lenalidomide (Revlimid®), thalidomide (Thalomid®); immune cell antibodies: alemtuzamab (Campath®), gemtuzumab (Myelotarg®), rituximab (Rituxan®), tositumomab (Bexxar®); interferons (e.g., IFN-alpha (Alferon®, Roferon-A®, Intron®-A) or IFN-gamma (Actimmune®)); interleukins: IL-1, IL-2 (Proleukin®), IL-24, IL-6 (Sigosix®), IL-12; HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”); anti-androgens which include, without limitation nilutamide (Nilandron®) and bicalutamide (Caxodex®); antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride; anti-hypercalcaemia agents which include without limitation gallium (III) nitrate hydrate (Ganite®) and pamidronate disodium (Aredia®); apoptosis inducers which include without limitation ethanol, 2-[[3-(2,3-dichlorophenoxy)propyl]amino]-(9C1), gambogic acid, elesclomol, embelin and arsenic trioxide (Trisenox®); Aurora kinase inhibitors which include without limitation binucleine 2; Bruton's tyrosine kinase inhibitors which include without limitation terreic acid; calcineurin inhibitors which include without limitation cypermethrin, deltamethrin, fenvalerate and tyrphostin 8; CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide; CD45 tyrosine phosphatase inhibitors which include without limitation phosphonic acid; CDC25 phosphatase inhibitors which include without limitation 1,4-naphthalene dione, 2,3-bis[(2-hydroxyethyl)thio]-(9C1); CHK kinase inhibitors which include without limitation debromohymenialdisine; cyclooxygenase inhibitors which include without limitation 1H-indole-3-acetamide, 1-(4-chlorobenzoyl)-5-methoxy-2-methyl-N-(2-phenylethyl)-(9C1), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid); cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylpheny1]-(9C1); cyclin dependent kinase inhibitors which include without limitation olomoucine and its derivatives, purvalanol B, roascovitine (Seliciclib®), indirubin, kenpaullone, purvalanol A and indirubin-3′-monooxime; cysteine protease inhibitors which include without limitation 4-morpholinecarboxamide, N-[(1S)-3-fluoro-2-oxo-1-(2-phenylethyl)propyl]amino]-2-oxo-1-(phenylmeth-yl)ethyl]-(9C1); DNA intercalators which include without limitation plicamycin (Mithracin®) and daptomycin (Cubicin®); DNA strand breakers which include without limitation bleomycin (Blenoxane®); E3 ligase inhibitors which include without limitation N-((3,3,3-trifluoro-2-trifluoromethyl)propionyl)sulfanilamide; EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980; farnesyltransferase inhibitors which include without limitation ahydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino]-4-(methylsulfony1)-1-methylethylester (2S)-(9C1), tipifarnib (Zarnestra®), and manumycin A; Flk-1 kinase inhibitors which include without limitation 2-propenamide, 2-cyano-3-[4-hydroxy-3,5-bis(1-methylethyl)phenyl]-N-(3-phenylpropyl)-(2E-)-(9C1); glycogen synthase kinase-3 (GSK3) inhibitors which include without limitation indirubin-3′-monooxime; histone deacetylase (HDAC) inhibitors which include without limitation suberoylanilide hydroxamic acid (SAHA), [4-(2-amino-phenylcarbamoyl)-benzyl]carbamic acid pyridine-3-ylmethylester and its derivatives, butyric acid, pyroxamide, trichostatin A, oxamflatin, apicidin, depsipeptide, depudecin, trapoxin, vorinostat (Zolinza®), and compounds disclosed in WO 02/22577; I-kappa B-alpha kinase inhibitors (IKK) which include without limitation 2-propenenitrile, 3-[(4-methylphenyl)sulfonyl]-(2E)-(9C1); imidazotetrazinones which include without limitation temozolomide (Methazolastone®, Temodar® and its derivatives (e.g., as disclosed generically and specifically in U.S. Pat. No. 5,260,291) and Mitozolomide; insulin tyrosine kinase inhibitors which include without limitation hydroxyl-2-naphthalenylmethylphosphonic acid; c-Jun-N-terminal kinase (JNK) inhibitors which include without limitation pyrazoleanthrone and epigallocatechin gallate; mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hy-droxyethyl)-4-methoxy-(9C1); MDM2 inhibitors which include without limitation trans-4-iodo, 4′-boranyl-chalcone; MEK inhibitors which include without limitation butanedinitrile, bis[amino[2-aminophenyl)thio]methylene]-(9C1); MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996; mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD; NGFR tyrosine kinase inhibitors which include without limitation tyrphostin AG 879; p38 MAP kinase inhibitors which include without limitation Phenol, 4-[4-(4-fluorophenyl)-5-(4-pyridinyl)-1H-imidazol-2-yl]-(9C1), and benzamide, 3-(dimethylamino)-N-[3-[(4-hydroxylbenzoyl)amino]-4-methylpheny1]-(9C1); p56 tyrosine kinase inhibitors which include without limitation damnacanthal and tyrphostin 46; PDGF pathway inhibitors which include without limitation tyrphostin AG 1296, tyrphostin 9, 1,3-butadiene-1,1,3-tricarbonitrile, 2-amino-4-(1H-indol-5-yl)-(9C1), imatinib (Gleevec®) and gefitinib (Iressa®) and those compounds generically and specifically disclosed in European Patent No.: 0 564 409 and PCT Publication No.: WO 99/03854; phosphatidylinositol 3-kinase inhibitors which include without limitation wortmannin, and quercetin dihydrate; phosphatase inhibitors which include without limitation cantharidic acid, cantharidin, and L-leucinamide; protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9C1) and benzylphosphonic acid; PKC inhibitors which include without limitation 1-H-pyrollo-2,5-dione, 3-[1-3-(dimethylamino)propyl]-1H-indol-3-yl]-4-(1H-indol-3-yl)-(9C1), Bisindolylmaleimide IX, Sphinogosine, staurosporine, and Hypericin; PKC delta kinase inhibitors which include without limitation rottlerin; polyamine synthesis inhibitors which include without limitation DMFO; PTP1B inhibitors which include without limitation L-leucinamide; protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587; SRC family tyrosine kinase inhibitors which include without limitation PP1 and PP2; Syk tyrosine kinase inhibitors which include without limitation piceatannol; Janus (JAK-2 and/or JAK-3) tyrosine kinase inhibitors which include without limitation tyrphostin AG 490 and 2-naphthyl vinyl ketone; retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®); RNA polymerase H elongation inhibitors which include without limitation 5,6-dichloro-1-beta-D-ribofuranosylbenzimidazole; serine/Threonine kinase inhibitors which include without limitation 2-aminopurine; sterol biosynthesis inhibitors which include without limitation squalene epoxidase and CYP2D6; VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

### EXAMPLES

Some aspects of the embodiments discussed above are disclosed in further detail in the following examples, which are not in any way intended to limit the scope of the present disclosure.

### Example 1

### Mitochondrial Transplantation Enhances Metabolic Fitness and Anti-Tumor Effects of CAR-T Cells

### Introduction

Chimeric antigen receptor (CAR) T cell therapy is a potentially curative treatment for patients with relapsed or refractory (r/r) hematopoietic malignancies. However, roughly half of patients with r/r B-cell lymphoma experience treatment failure after CAR-T cell therapy possibly due to poor expansion, limited persistence, and exhaustion of CAR-T cells. Given that metabolic fitness has a central role in regulating T cell functions, manipulating the mitochondrial function is a promising approach to enhance anti-tumor effects of CAR-T cells. Recently, it has been shown that transfer of live mitochondria (mitochondrial transplantation) prepared by the conventional homogenization method (Mito-conv) into T cells enhanced the expression of inhibitory receptors and reduced T cell proliferation (Court AC: EMBO Rep 2020). In the current study, it was tested if transfer of “Q” mitochondria isolated from Hela cells using a novel iMIT isolation technology (WO/2021/015298 A1) into T cells could enhance metabolic fitness and anti-tumor effects of CAR-T cells.

### Methods

Purified T cells from naïve mice or healthy volunteers were stimulated with plate-bound anti-CD3/-CD28 antibodies in the presence or absence of 50-100 μg/ml Q mitochondria. Culture medium and Q- were renewed every 24 or 48 hours. Mitochondrial respiratory function was evaluated using the XFp Flux Analyzer. For CAR-T cell generation, purified T cells were stimulated with anti-CD3/-CD28 antibodies for 48 hrs and then incubated with retroviral vector encoding anti-CD19 CAR (1D3-28Z.1-3; Addgene) for another 48 hrs.

### Results

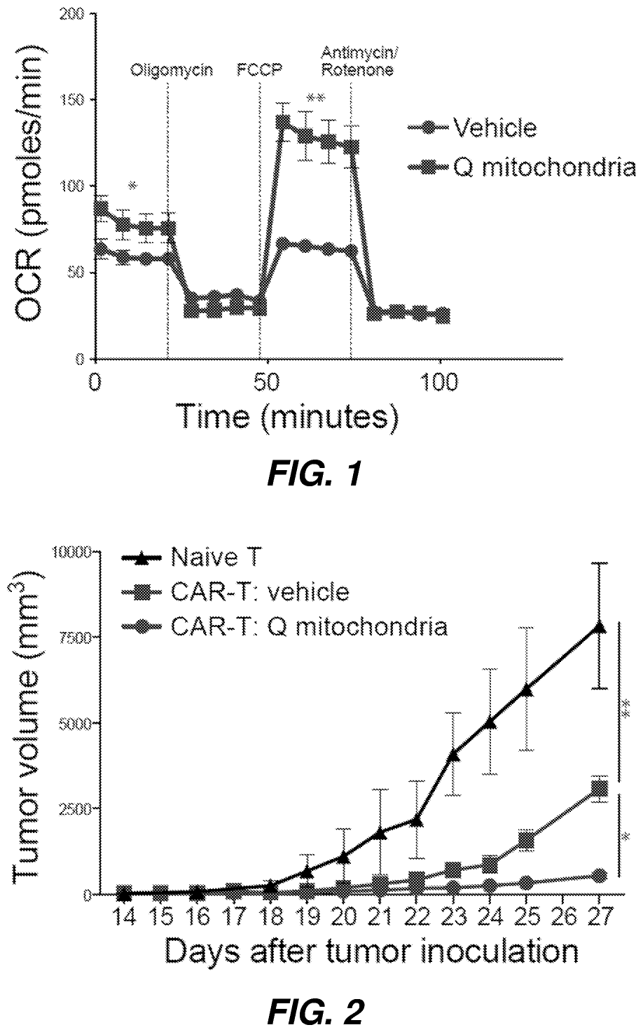

First, it was confirmed that mitochondrial structure and ATP synthesis of Q mitochondria were much better maintained compared to those of Mito-conv. When mouse purified T cells were incubated with fluorescent-labeled Q mitochondria for 24 hrs during TCR stimulation, approx. 70% of T cells endocytosed Q mitochondria, and mitochondrial mass per cell was significantly increased. As expected, oxidative phosphorylation (OXPHOS) was markedly enhanced in Q-treated T cells compared to vehicle-treated controls after TCR stimulation. The levels of reactive oxygen species (ROS) in vehicle-treated T cells were increased after 72-hrs TCR stimulation, while Q mitochondria significantly mitigated accumulation of ROS in activated T cells. Q mitochondria suppressed differentiation of terminally exhausted T cells and increased the TCF-1⁺ precursor population for memory and exhausted T cells (e.g., TCF-1⁺PD-1⁺Tim3-precursor exhausted T cells (pTex)) in association with improved survival and enhanced production of IFN-g and TNF-a after chronic TCR stimulation (196 hrs). Based on these findings, it was next investigated whether Q mitochondria could affect CAR-T cell functions. CAR-T cells were generated with purified T cells from naïve BALB/c mice and Q mitochondria was added from the beginning of TCR stimulation during the CAR-T cell generation. Q-treated CAR-T cells demonstrated enhanced OXPHOS and cytokine production compared to vehicle-treated CAR-T cells (FIG. 1). When CAR-T cells were incubated with syngeneic B-cell lymphoma cells (A20) for 12 hrs, Q-treated CAR-T cells demonstrated significantly enhanced in vitro cytotoxicity against A20 lymphoma cells. Next, in vivo antitumor effects of CAR-T cells was examined. Naïve BALB/c mice were subcutaneously injected with 15×10⁶ A20 cells on the right flank and intravenously injected with 1×10⁶ Q-treated or control CAR-T cells, or naïve BALB/c T cells 14 days after tumor inoculation. Although control CAR-T cells significantly suppressed tumor growth compared to naïve T cells, all mice died due to tumor growth by day 31 after tumor inoculation. Importantly, tumor growth was further suppressed in the mice injected with Q-treated CAR-T cells leading to significantly prolonged survival (FIG. 2).

### Conclusions

For the first time it was found CAR-T cells transplanted with Q mitochondria prepared with the iMIT method improved metabolic fitness, leading to enhanced proliferative capacity and cytokine production, and in vitro and in vivo anti-tumor effects. Mitochondrial transplantation could be also useful as an adjunctive method in combination with various other genetical and pharmacological approaches to enhance effectiveness of CAR-T cell therapy.

### Example 2

### Modulation of T Cell Function With Second Organelle Complexes Transplantation

Naïve T cells depend on oxidative phosphorylation (OXPHOS) for energy production, whereas activated T cells show an increase in overall metabolic activities such as glycolysis and glutamate degradation. In situations where T cells receive chronic antigen stimulation, such as in the tumor microenvironment, reactive oxygen species (ROS) accumulate, and mitochondrial function is impaired. Mitochondrial dysfunction and decreased mitochondrial mass can induce decreased production of effector cytotoxic cytokines in T cells. Additionally, ROS accumulation in mitochondria inhibits OXHPOS and induces T cell exhaustion. CAR-T cells from patients who failed CAR-T therapy can show decreased mitochondria neogenic potential and reduced mitochondrial mass, which was associated with decreased proliferative capacity and persistence. In some embodiments, second organelle complexes transplantation at the time of CAR-T generation improves the efficacy of CAR-T therapy. This example demonstrates that transfer of second organelle complexes improves T cell function by restoring mitochondrial energy production and reducing mitochondrial ROS.

Second organelle complexes were isolated from Hela cells. After cell membrane permeabilization with digitonin and a wash out step, cells were incubated on ice. This was followed by pipetting and centrifugation to retrieve isolated second organelle complexes.

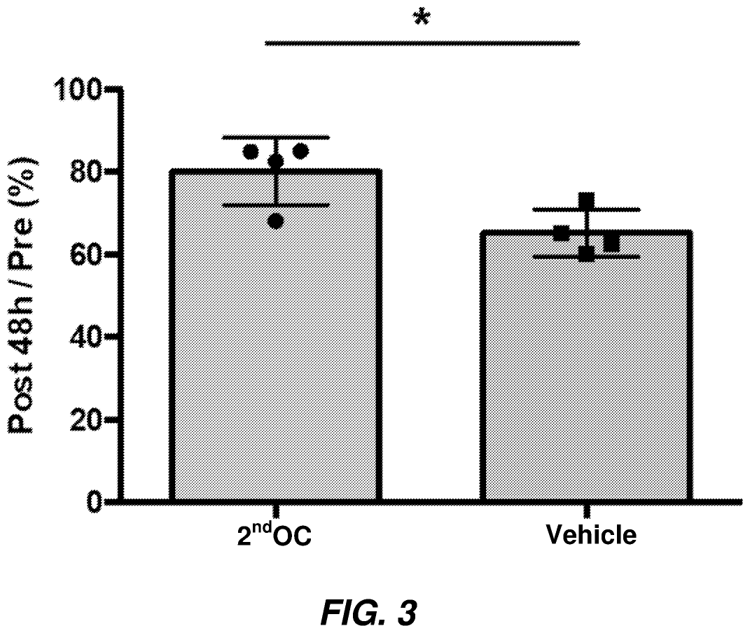

Second organelle complexes administration was found to improve the efficiency of T cell recovery after stimulation. FIG. 3 depicts data related to the efficiency of T cell recovery after stimulation. Transplantation of second organelle complexes (or vehicle) was performed at day 1 followed by stimulation with anti-CD3/CD28 antibodies. T cell recovery was measured 48 hours later. T cell recovery efficiency (%) is defined as {(Number of recovered T cells after 48 hours of stimulation)/(Number of T cells immediately before the start of stimulation)}×100.

The effect of second organelle complexes transplantation on chronically stimulated T cells was examined. Chronic T cell stimulation was performed to reproduce the tumor microenvironment. T cell purification (negative selection on MACS) from C57BL/6 mice was followed by T cell stimulation (via plate bound anti-CD3/CD28 Abs) at day 1. Second organelle complexes administration was performed at days 1, 3, 5, and 7 (or vehicle for control) followed by FACS analysis at day 4 or 9.

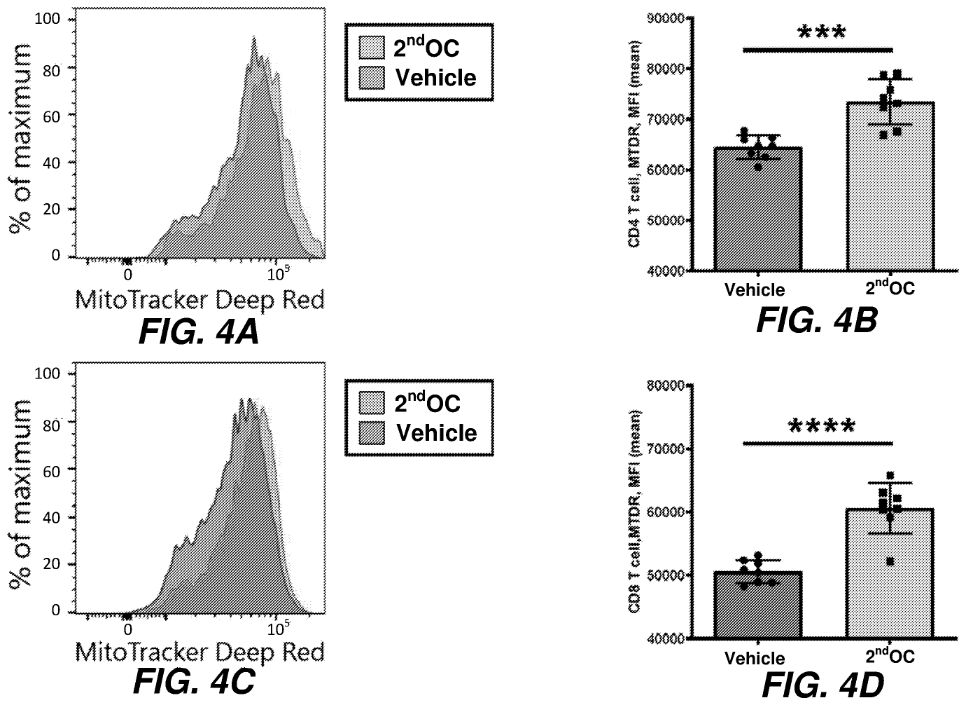

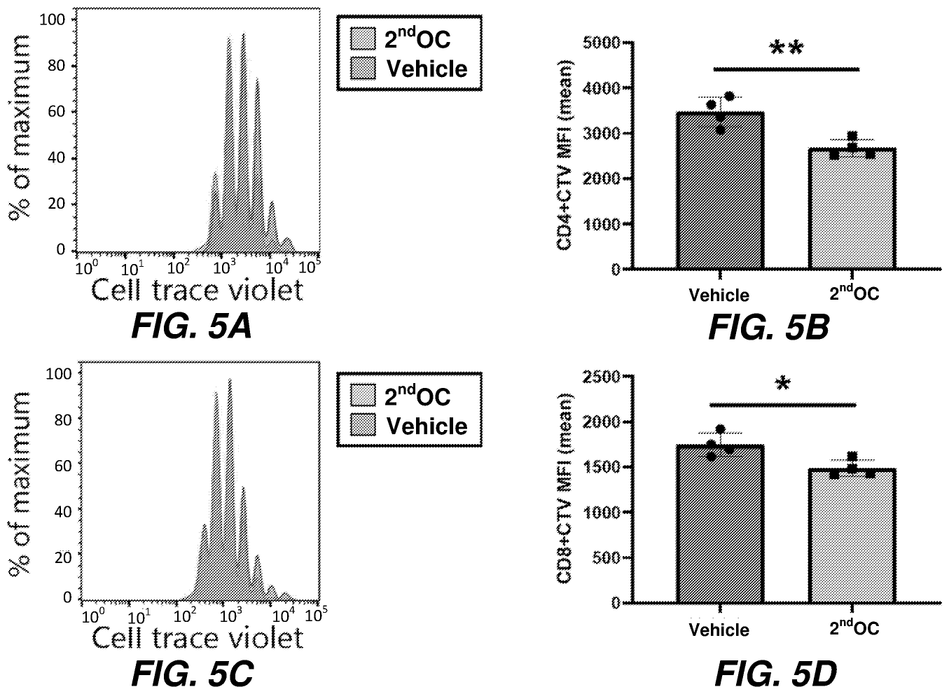

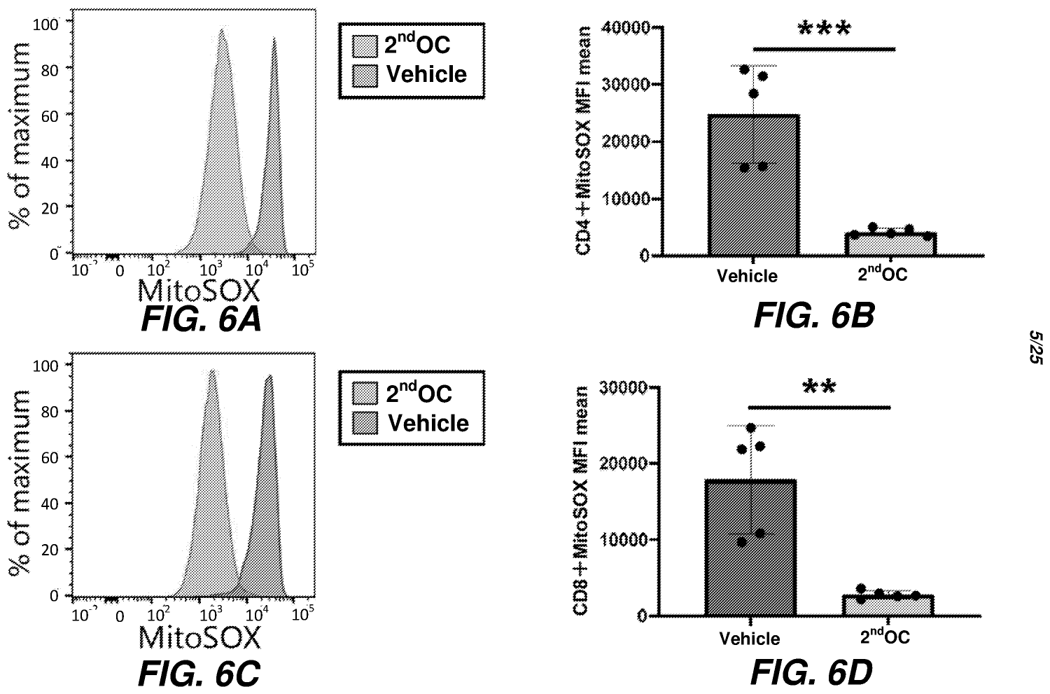

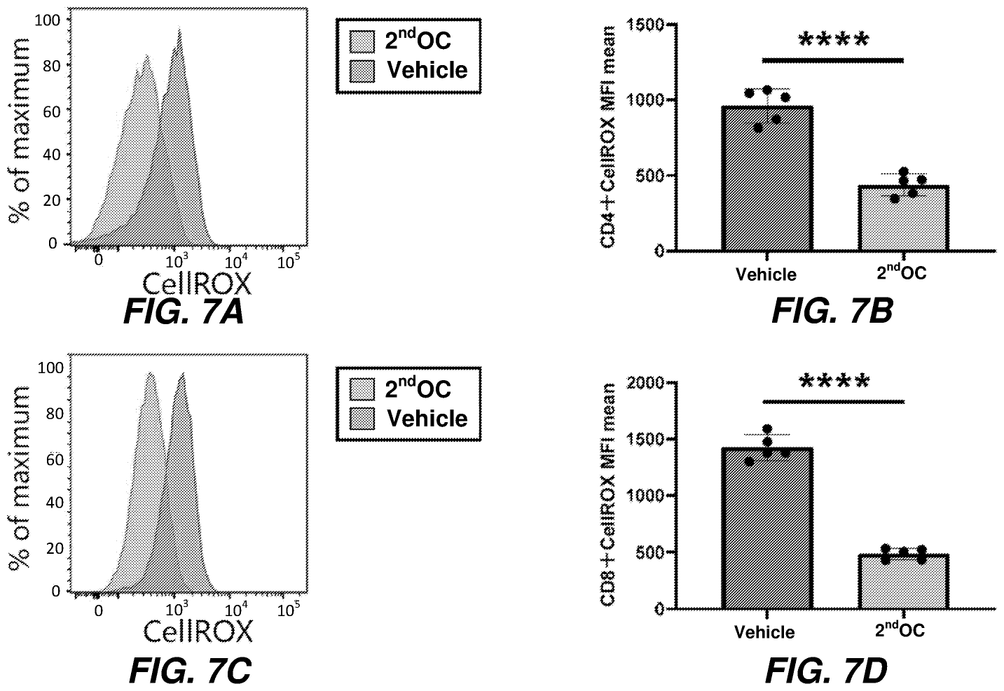

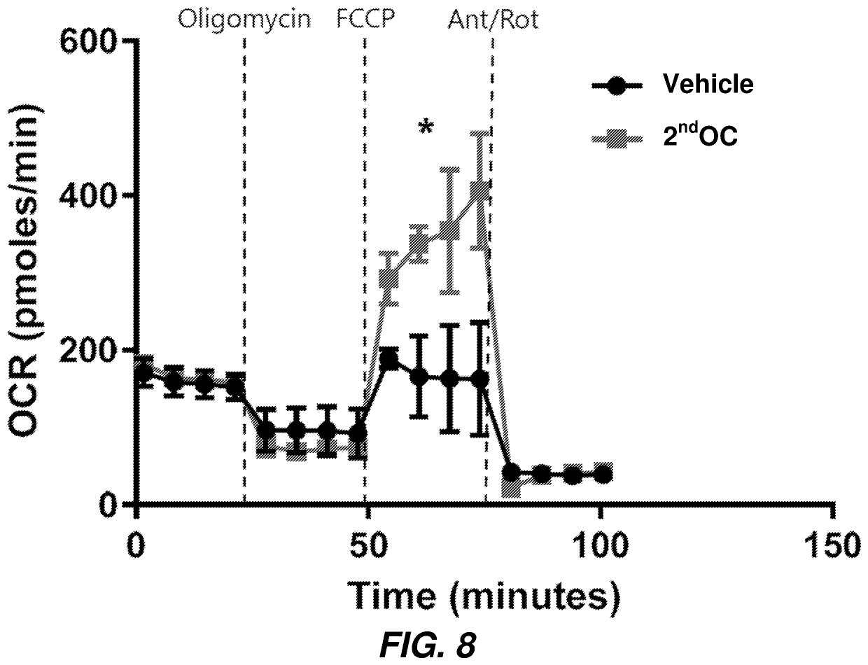

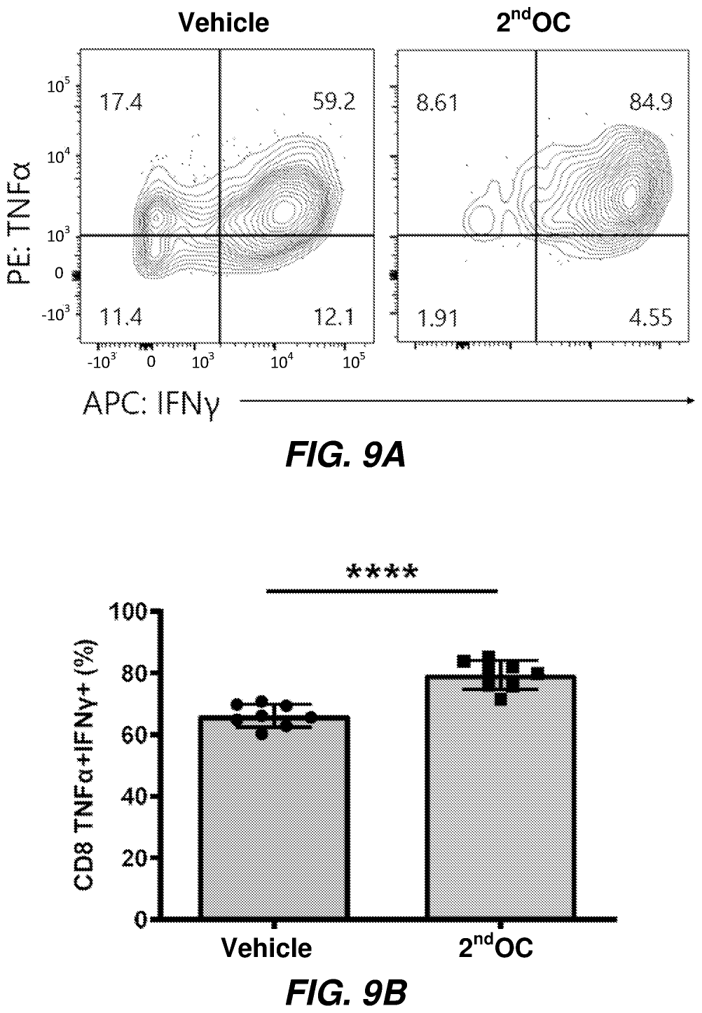

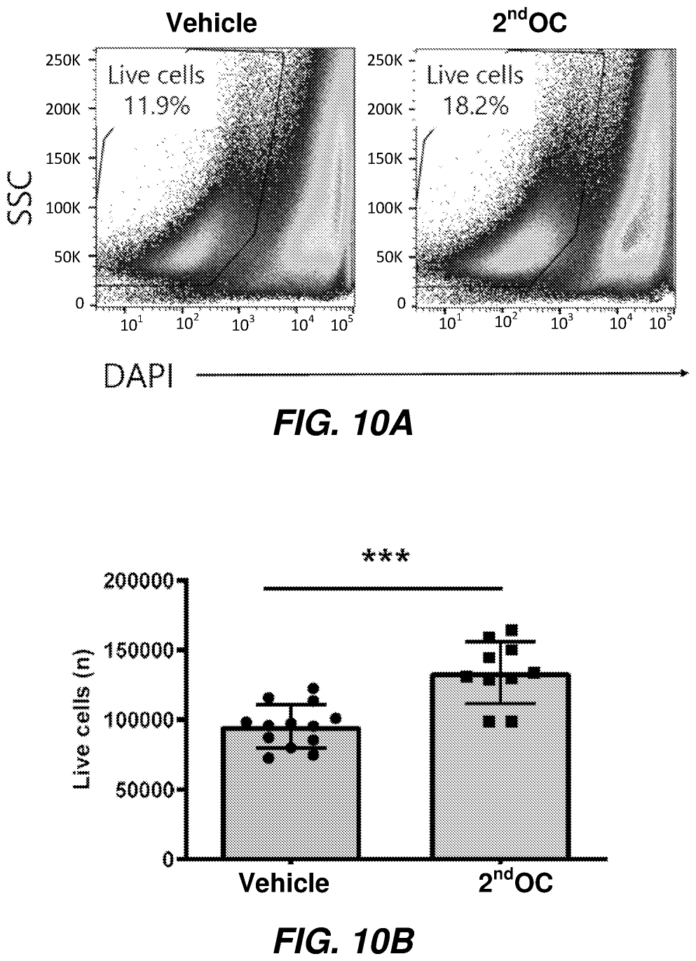

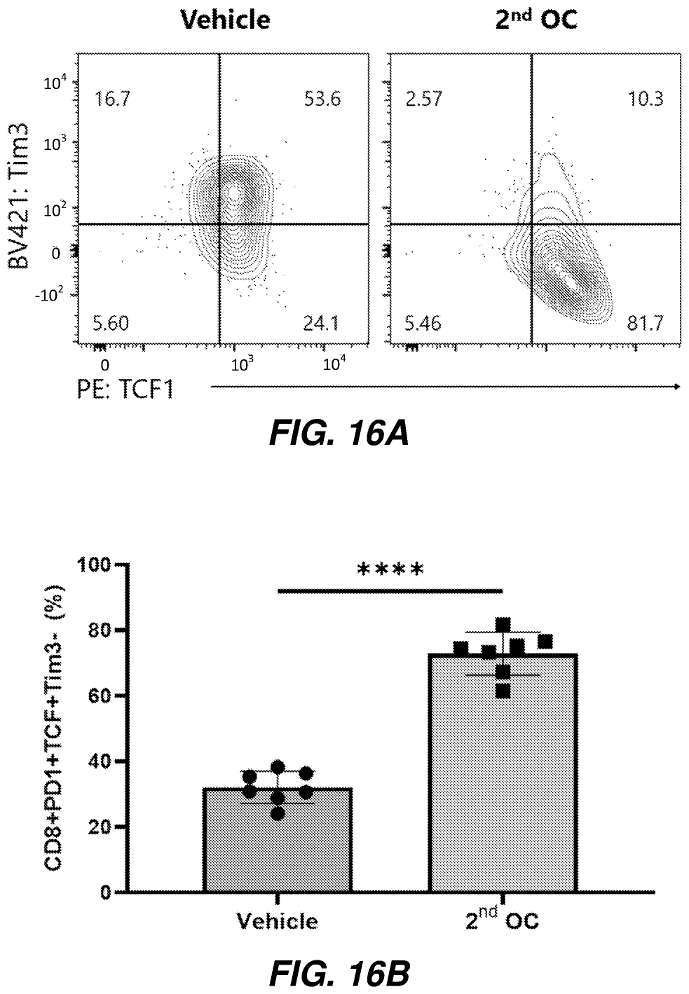

Second organelle complexes transplantation was found to increase T cell mitochondrial mass (FIGS. 4A-4D). Next, the effect of second organelle complexes transplantation on T cell proliferative capacity was examined. FIGS. 5A-5D depict data related to T cell proliferation capacity assessed by a dye dilution assay using Cell Trace Violet dye. CD4 T cells (FIGS. 5A-5B) and CD8 T cells (FIGS. 5C-5D) were transplanted with second organelle complexes or vehicle and assessed at day 4. Second organelle complexes transplantation was found to improve T cell proliferative capacity. The effect of second organelle complexes transplantation on mitochondrial ROS levels was next assayed, and second organelle complexes transplantation was found to reduce ROS levels accumulated in mitochondria (FIGS. 6A-6D). Second organelle complexes transplantation was also found to reduce the accumulation of ROS levels throughout the T cell (FIGS. 7A-7D). The effect of second organelle complexes transplantation on T cell mitochondrial respiratory capacity was next assayed. FIG. 8 depicts data related to T cell oxygen consumption rate measured with a XFp Flux Analyzer. T cells were transplanted with second organelle complexes or vehicle and assessed at day 4. Second organelle complexes transplantation was found to improve T cell mitochondrial respiratory capacity. Second organelle complexes transplantation was found to enhance the cytokine-producing capacity of CD8 T cells (FIGS. 9A-9B). FIGS. 10A-10B depict data related to the number of surviving cells after in vitro TCR chronic stimulation. T cells were transplanted with second organelle complexes or vehicle and assessment of viable cell count was performed using a dead cell staining dye at Day 9. Second organelle complexes transplantation was found to increase the number of viable T cells after chronic stimulation. Flow cytometric analysis of TCF-1 precursor cells was performed after 72 hours of TCR stimulation to compare T cells transplanted with second organelle complexes (2^{nd} OC) or vehicle (FIGS. 16A-16B).

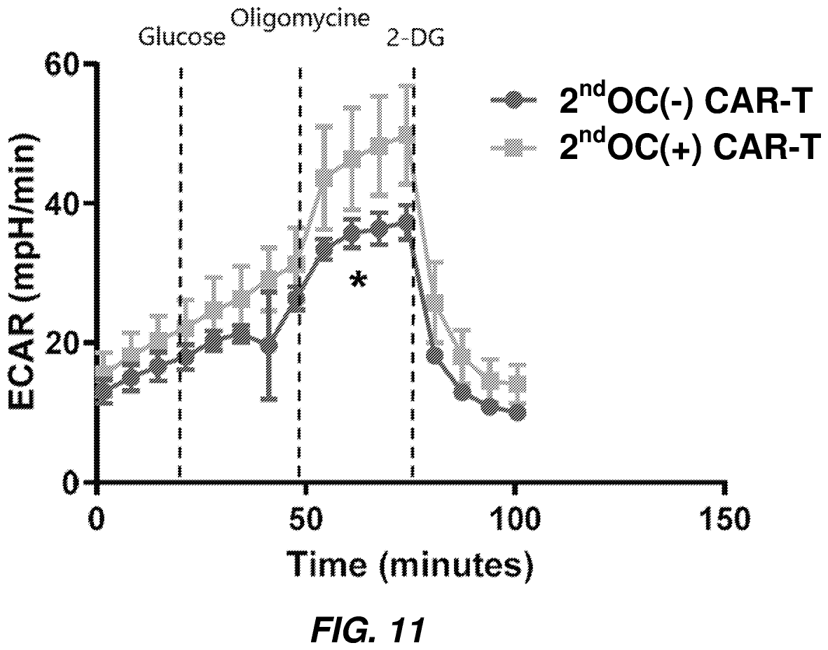

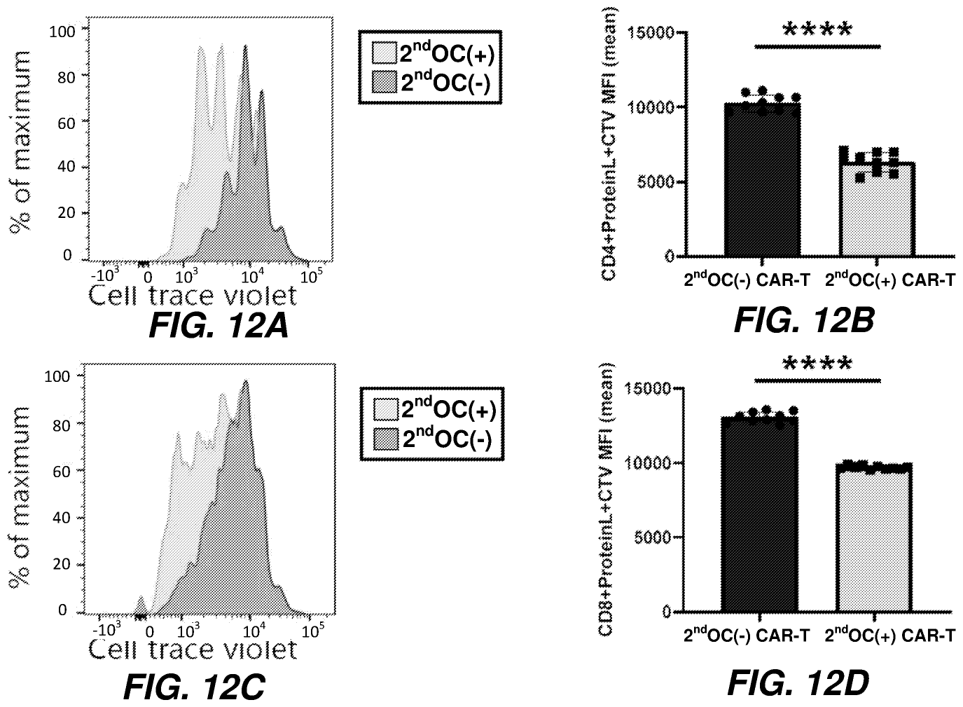

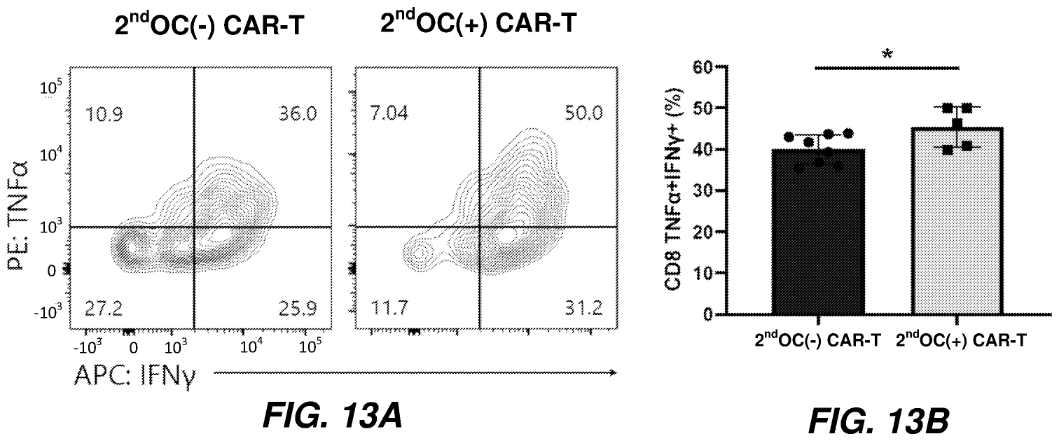

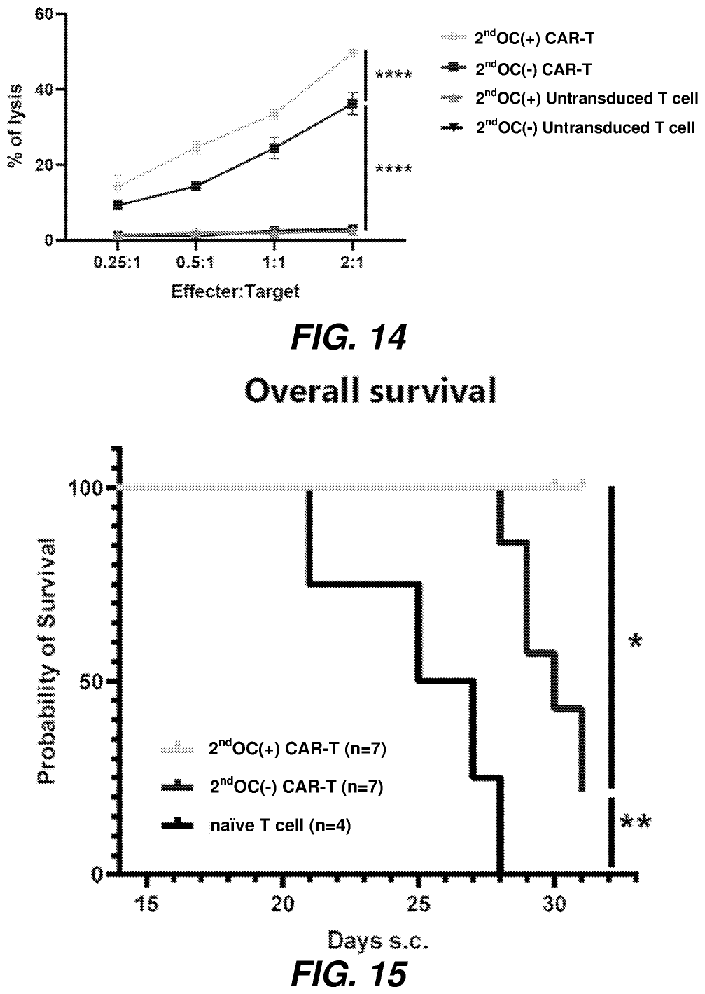

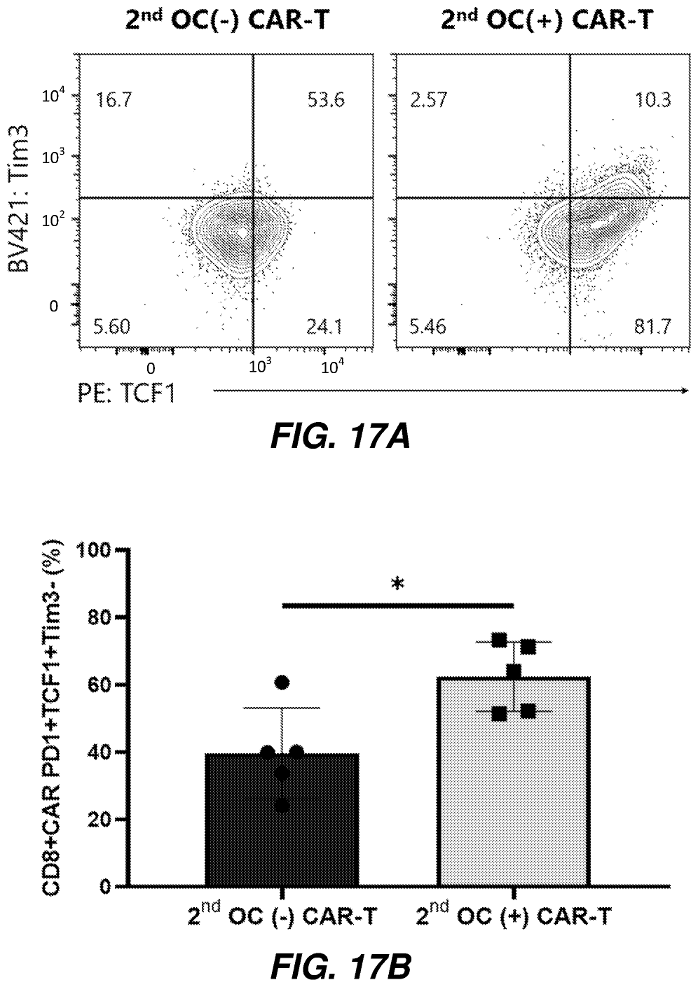

Mouse CD19 CAR-T generation was performed as follows. T cell purification (negative selection on MACS) from BALB/c mice was followed by T cell stimulation at Day 1 (plate bound anti-CD3/CD28 antibodies) for 48 hours. A vector plasmid (CD19 CAR) comprising 1D3 scFv, CD28, and CD3ζ 1st and 3rd ITAMs inactive was used to generate (via transfection) retrovirus particles. CAR gene transduction was performed at days 3 and 4 (with the retroviral vector). Second organelle complexes were administered three times (at days 1, 3, and 4). FIG. 11 depicts data related to CAR-T cell glycolytic rates measured with a Seahorse Extracellular Flux Analyzer. FIG. 1 depicts CAR-T cell oxygen consumption rates measured with a Seahorse Extracellular Flux Analyzer following T cell transplantation with second organelle complexes or vehicle. Second organelle complexes transplantation was found to improve T cell mitochondrial respiratory capacity and glycolytic capacity. Second organelle complexes transplantation was found to improve CAR-T cell proliferative capacity (FIGS. 12A-12D). Next, cytokine production capacity was evaluated. Second organelle complexes transplantation was found to enhance the cytokine-producing capacity of CD8 CAR-T cells (FIGS. 13A-13B). Second organelle complexes transplantation was also found to enhance the cytotoxic activity of CAR-T cells in vitro (FIG. 14). Flow cytometric analysis of TCF-1 precursor CAR-T cells was performed 96 hours after the initiation of TCR stimulation to compare CAR-T cells transplanted with second organelle complexes (2^{nd} OC) or vehicle (FIGS. 17A-17B).

An in vivo evaluation of antitumor effects of CAR-T was next performed using a B cell lymphoma model (A20, 15×10⁶ cells s.c.). 1×10⁶ cells were injected intravenously into BALB/c mice bearing A20 tumor at day 14, with 2^{nd}OC(+) CAR-T cells (n=7), 2^{nd}OC(−) CAR-T cells (n=7), and naïve T cells (n=4) being compared. FIG. 2 depicts data related to the suppression of tumor growth in the mice injected with second organelle complexes-transplanted CAR-T cells, leading to significantly prolonged survival. FIG. 15 depicts data related to overall survival of mice injected with second organelle complexes-transplanted CAR-T cells as compared to vehicle-transplanted CAR-T cells or naïve T cells. Second organelle complexes transplantation was found to enhance the anti-tumor effect of CAR-T and improved survival.

This example demonstrates that transplantation of second organelle complexes during in vitro TCR chronic stimulation improved the proliferative capacity of T cells and increased the number of surviving cells. By transplanting second organelle complexes, cellular and mitochondrial ROS levels decreased while mitochondrial respiratory capacity improved. Additionally, transplantation of second organelle complexes during the production of CD19 CAR-T from naïve T cells improved the T cell recovery efficiency after anti-CD3/CD28 antibody stimulation. Importantly, second organelle complexes transplantation improved the mitochondria respiratory capacity of mouse CD19 CAR-T and improved the antitumor effect.

### Example 3

### Study of Impact of Organelle Complexes Transplantation on Metabolic Fitness and Anti-Tumor Effects of CAR-T Cells

### Introduction

In this study, it will be tested if transfer of organelle complexes (e.g., first organelle complexes, second organelle complexes) isolated from Hela cells into T cells will enhance metabolic fitness and anti-tumor effects of CAR-T cells.

### Methods

Purified T cells from naïve mice or healthy volunteers will be stimulated with plate-bound anti-CD3/-CD28 antibodies in the presence or absence of 50-100 μg/ml of organelle complexes (e.g., first organelle complexes, second organelle complexes). Culture medium and organelle complexes (e.g., first organelle complexes, second organelle complexes) will be renewed every 24 or 48 hours. Mitochondrial respiratory function will be evaluated using the XFp Flux Analyzer. For CAR-T cell generation, purified T cells will be stimulated with anti-CD3/-CD28 antibodies for 48 hrs and then incubated with retroviral vector encoding anti-CD19 CAR (1D3-28Z.1-3; Addgene) for another 48 hrs.

### Expected Results

First, it will be confirmed that mitochondrial structure and ATP synthesis of T cells transplanted with organelle complexes (e.g., first organelle complexes, second organelle complexes) are much better maintained compared to those of Mito-conv. Mouse purified T cells will be incubated with fluorescent-labeled organelle complexes (e.g., first organelle complexes, second organelle complexes) for 24 hrs during TCR stimulation, and it is expected that approx. 70% of T cells will have endocytosed organelle complexes, and mitochondrial mass per cell will be significantly increased. Oxidative phosphorylation (OXPHOS) will be markedly enhanced in organelle complexes (OC)-treated T cells compared to vehicle-treated controls after TCR stimulation. The levels of reactive oxygen species (ROS) in vehicle-treated T cells will be increased after 72-hrs TCR stimulation, while organelle complexes (e.g., first organelle complexes, second organelle complexes) will significantly mitigate accumulation of ROS in activated T cells. Organelle complexes (e.g., first organelle complexes, second organelle complexes) will suppress differentiation of terminally exhausted T cells and will increase the TCF-1⁺ precursor population for memory and exhausted T cells (e.g., TCF-1⁺PD-1⁺Tim3-precursor exhausted T cells (pTex)) in association with improved survival and enhanced production of IFN-g and TNF-a after chronic TCR stimulation (96 hrs). It will next be investigated whether organelle complexes (e.g., first organelle complexes, second organelle complexes) can affect CAR-T cell functions. CAR-T cells will be generated with purified T cells from naïve BALB/c mice and organelle complexes (e.g., first organelle complexes, second organelle complexes) will be added from the beginning of TCR stimulation during the CAR-T cell generation. OC-treated CAR-T cells will demonstrate enhanced OXPHOS and cytokine production compared to vehicle-treated CAR-T cells. CAR-T cells will be incubated with syngeneic B-cell lymphoma cells (A20) for 12 hrs, and OC-treated CAR-T cells will demonstrate significantly enhanced in vitro cytotoxicity against A20 lymphoma cells. Next, in vivo antitumor effects of CAR-T cells will be examined. Naïve BALB/c mice will be subcutaneously injected with 1.5×10⁷ A20 cells on the right flank and will be intravenously injected with 1×10⁶ OC-treated or control CAR-T cells, or naïve BALB/c T cells 14 days after tumor inoculation. It is expected that although control CAR-T cells will significantly suppress tumor growth compared to naïve T cells, all mice will die due to tumor growth by day 31 after tumor inoculation. Tumor growth will be further suppressed in the mice injected with OC-treated CAR-T cells leading to significantly prolonged survival.

### Expected Conclusions

CAR-T cells transplanted with organelle complexes (e.g., first organelle complexes, second organelle complexes) will exhibit improved metabolic fitness, which will lead to enhanced proliferative capacity and cytokine production, and in vitro and in vivo anti-tumor effects. Organelle complexes transplantation could be also useful as an adjunctive method in combination with various other genetical and pharmacological approaches to enhance effectiveness of CAR-T cell therapy.

### Example 4

### Organelle Complexes Transplantation Suppresses Ferroptosis in T Cells

### Introduction

This Example investigates the impact of organelle complexes (e.g., first organelle complexes, second organelle complexes (Q)) transplantation on ferroptosis in T cells. Ferroptosis is a unique form of regulated cell death that can cause an enormous loss of antigen-specific T cells, including virus-specific CD4⁺ helper T cells (See Wang, Yifei, et al. “The kinase complex mTORC2 promotes the longevity of virus-specific memory CD4⁺ T cells by preventing ferroptosis.” Nature immunology 23.2 (2022): 303-317). Ferroptosis can be driven by an iron-dependent lethal accumulation of membrane lipid peroxidation and can occur under quiescent conditions when the polyunsaturated fatty acid (PUFA) tails of membrane phospholipids become excessively peroxided into toxic lipid peroxides (PL-PUFA-OOH). Mitochondria are responsible for the generation of the majority of endogenous ROS, such as superoxide anion and hydrogen peroxide, and both of these species can act as the substrates of the Fenton reaction to yield hydroxyl radicals, which in turn can promote the propagation of phospholipid peroxidation and degradation of membrane lipids. In contrast, GPX4, a glutathione-dependent peroxidase, can convert toxic PL-PUFA-OOH to non-toxic PL-PUFA-OH propelled by oxidizing glutathione (GSH) to glutathione disulfide (GSSG), thereby suppressing membrane lipid hydroperoxides. Transplantation of second organelle complexes (Q) was hypothesized to suppresses ferroptosis and improve T cell survival.

### Q Transplantation Promotes the Expression of GPX4 in Activated T Cells

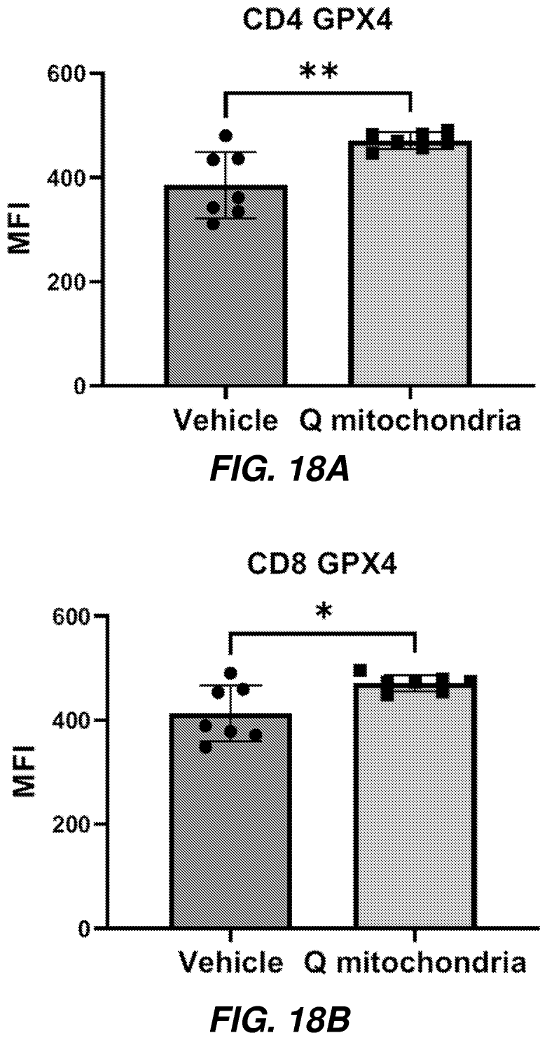

FIGS. 18A-18B depict data related to GPX4 expression in CD4⁺ T cells (FIG. 18A) and CD8⁺ T cells (FIG. 18B) transplanted with second organelle complexes (Q) or vehicle after 72 hours stimulation with anti-CD3/CD28 antibodies. It was found that Q transplantation promoted the expression of GPX4, a molecule that suppresses ferroptosis.

### Q Transplantation Suppresses Lipid Peroxidation in Activated T Cells

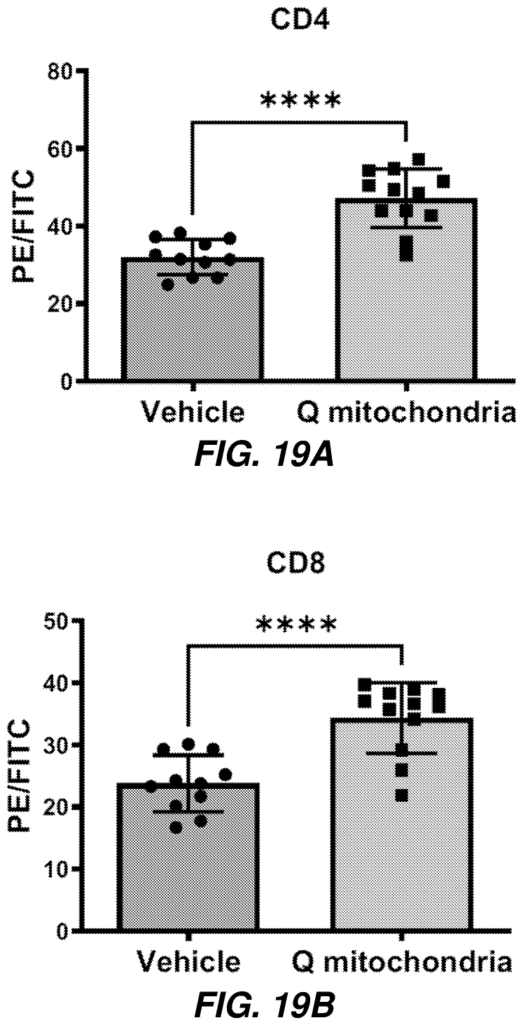

FIGS. 19A-19B depict data related to lipid peroxidation levels in CD4⁺ T cells (FIG. 19A) and CD8⁺ T cells (FIG. 19B) transplanted with second organelle complexes (Q) or vehicle after 72 hours stimulation with anti-CD3/CD28 antibodies. Lipid peroxidation was evaluated as the fluorescence change from PE to FITC using a lipid peroxidation kit. As the ratio of PE/FITC increases, the level of lipid peroxidation decreases. These results show that second organelle complexes transplantation is capable of suppressing peroxidation of lipids.

### Q Transplantation Suppresses Cell Death of Activated T Cells

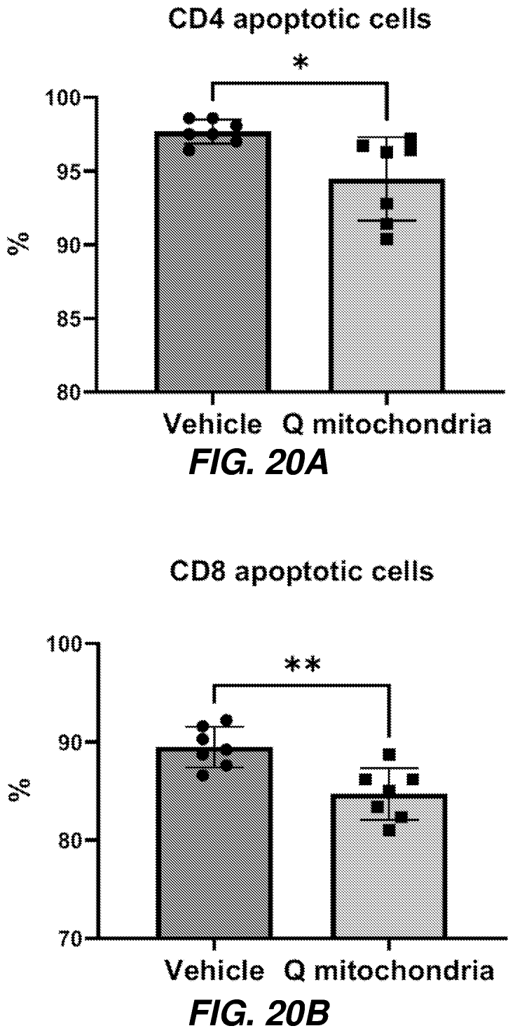

FIGS. 20A-20B depict data related to the percentage of apoptotic CD4⁺ T cells (FIG. 20A) and CD8⁺ T cells (FIG. 20B) transplanted with second organelle complexes (Q) or vehicle after 120 hours stimulation with anti-CD3/CD28 antibodies. It was found that second organelle complexes transplantation is capable of suppressing cell death of activated T cells, which can be considered ferroptosis suppression.

### Q Transplantation Suppresses ROS Accumulation in Mouse CAR-T Cells

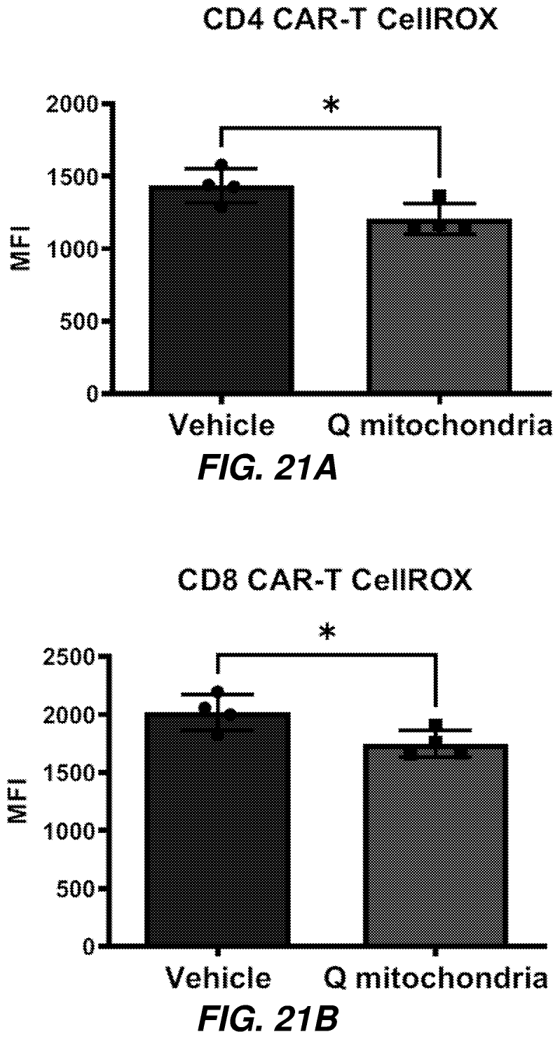

FIGS. 21A-21B depict data related to ROS accumulation of Vehicle CAR-T cells and Q-treated CAR-T cells assessed with CellROX Green dye in a CD4⁺ (FIG. 21A) and CD8⁺ (FIG. 21B) context. A marked reduction in ROS accumulation was found in CAR-T cells transplanted with second organelle complexes.

### Q Transplantation Suppresses Lipid Peroxidation in Mouse CAR-T Cells

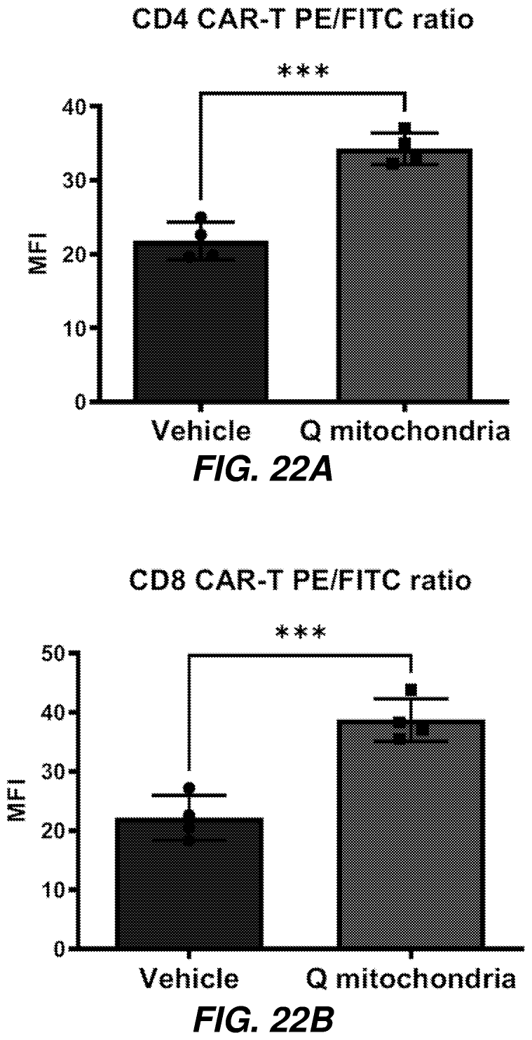

FIGS. 22A-22B depict data related to lipid peroxidation levels in CD4⁺ CAR-T cells (FIG. 22A) and CD8⁺ CAR-T cells (FIG. 22B) transplanted with second organelle complexes (Q) or vehicle. Lipid peroxidation was evaluated as the fluorescence change from PE to FITC using a lipid peroxidation kit. As the ratio of PE/FITC drops, the level of lipid peroxidation increases. Second organelle complexes transplantation was found to suppress the peroxidation of lipids.

### Q Transplantation of Mouse CAR-T Cells Prolongs Survival in Tumors After Infusion

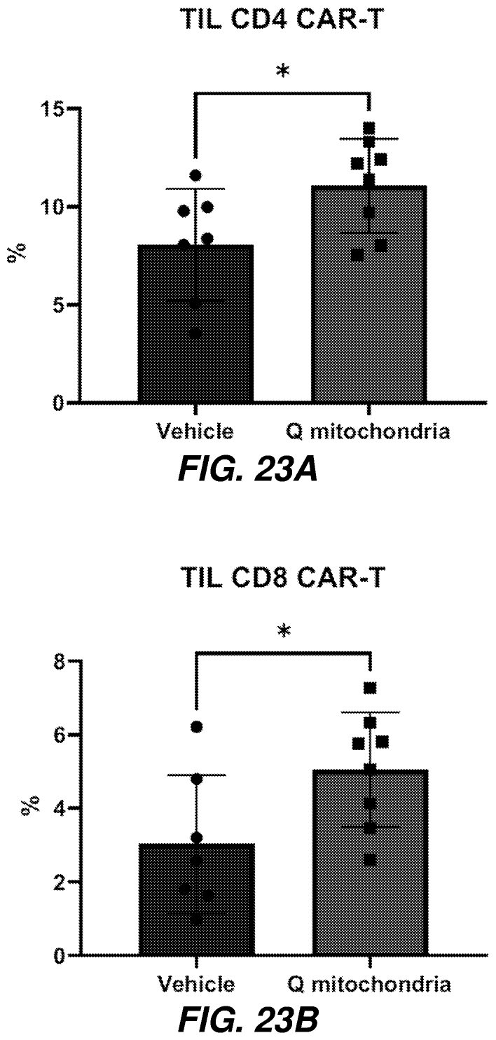

FIGS. 23A-23B depict data related to the percentage of CD4⁺ CAR-T cells (FIG. 23A) and CD8⁺ CAR-T cells (FIG. 23B) transplanted with second organelle complexes (Q) or vehicle in tumor infiltrating lymphocytes (TIL) at 17 days after tumor cell inoculation. An increased percentage in both CD4⁺ and CD8⁺ CAR-T cells was found in tumors in the second organelle complexes transplantation group.

### Q Transplantation Suppresses ROS Accumulation in Human Activated T Cells

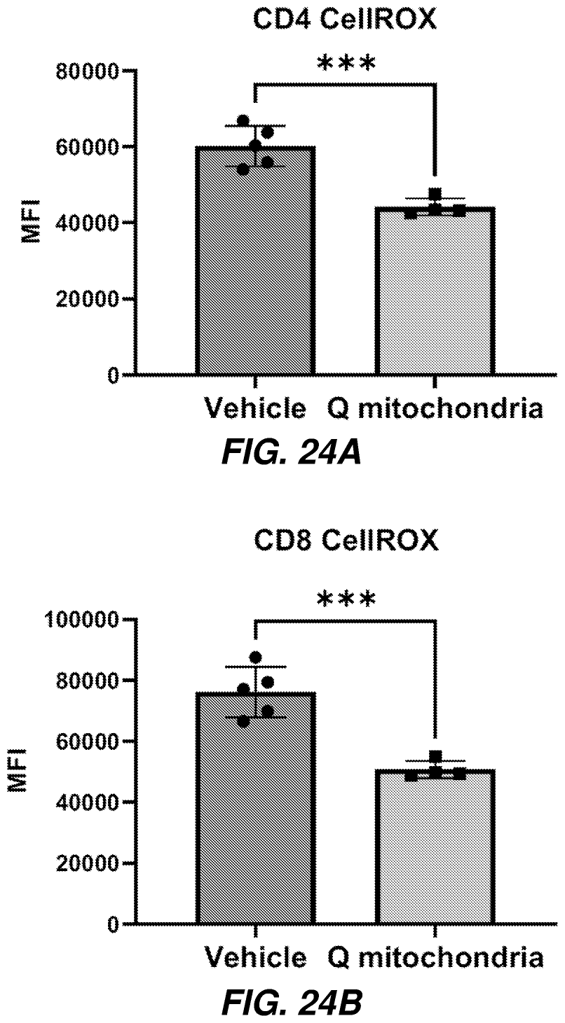

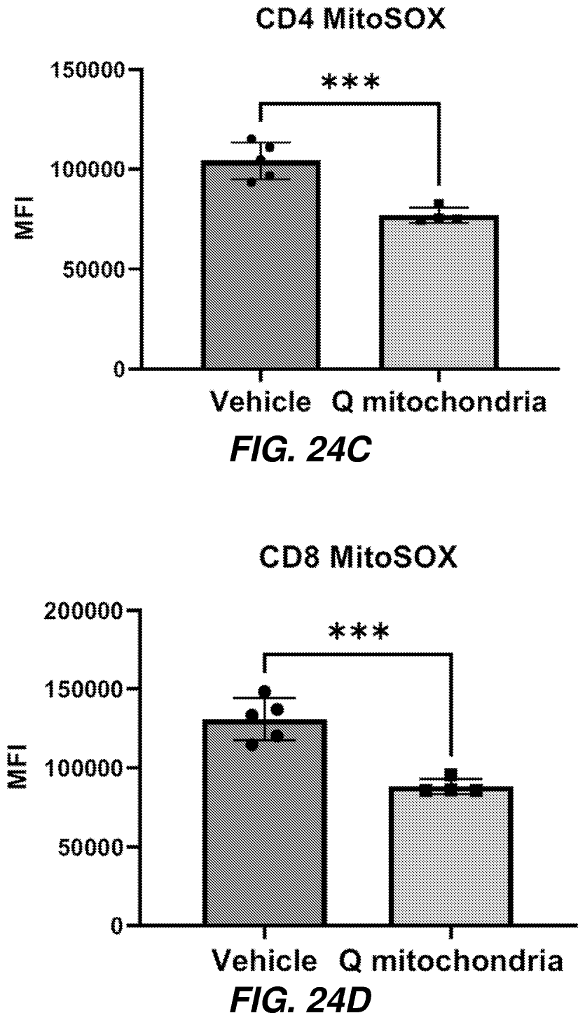

FIGS. 24A-24D depict data related to cellular ROS (FIGS. 24A-24B) and mitochondrial ROS (FIGS. 24C-24D) accumulation in CD4⁺ T cells (FIG. 24A, FIG. 24C) and CD8⁺ T cells (FIG. 24B, FIG. 24D) transplanted with second organelle complexes (Q) or vehicle after 192 hours stimulation with anti-CD3/CD28 antibodies. Cellular ROS and mitochondrial ROS accumulation was assessed using CellROX Green dye and MitoSOX Red dye, respectively. Second organelle complexes transplantation of human activated T cells was found to exert a suppressive effect on ROS accumulation.

### Q Transplantation Promotes Proliferation of Human Activated T Cells

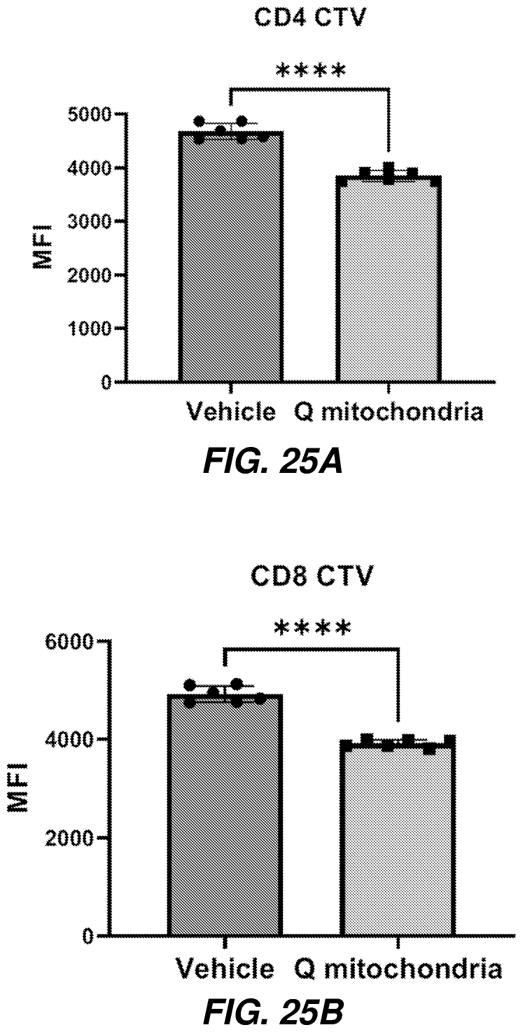

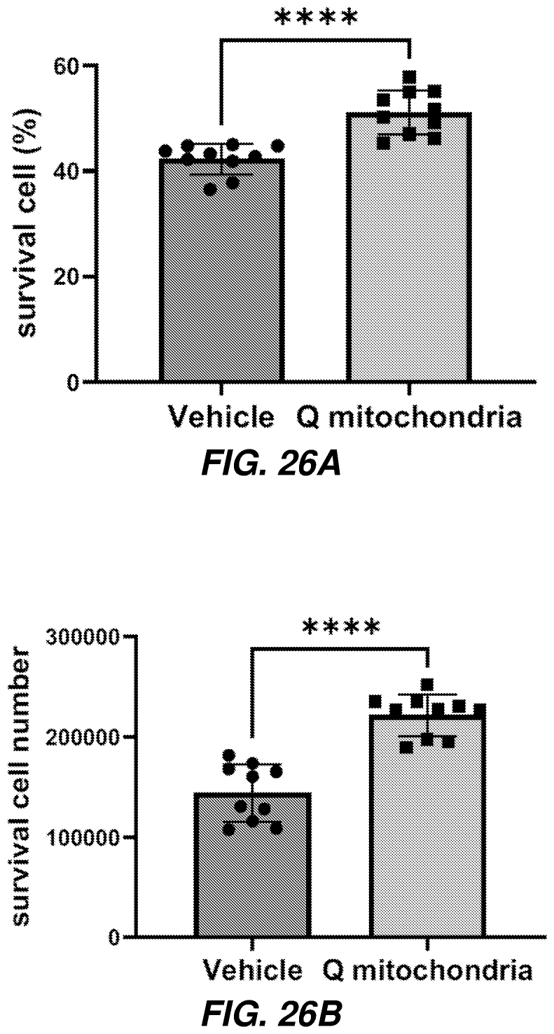

FIGS. 25A-25B depict data related to the proliferation ability of CD4⁺ T cells (FIG. 25A) and CD8⁺ T cells (FIG. 25B) transplanted with second organelle complexes (Q) or vehicle after chronic stimulation. T cell proliferation ability was assessed using Cell Trace Violet dilution assay. An assessment of the proportion and number of viable T cells using dead cell staining dye was also conducted after 192 hours stimulation with anti-CD3/-CD28 antibodies. FIGS. 26A-26B depict data related to viable cell percentage (FIG. 26A) and viable cell number (FIG. 26B) of T cells transplanted with second organelle complexes (Q) or vehicle after 192 hrs stimulation with anti-CD3/-CD28 antibodies. Second organelle complexes transplantation was found to enhance the proliferation of human activated T cells.

### Conclusions

Ferroptosis is an important apoptosis pathway in T cells linked to mitochondrially generated ROS. In a variety of models tested (including CD4⁺ T cells and CD8⁺ T cells in human, mouse, and CAR-T contexts) second organelle complexes (Q) transplantation was demonstrated to promote GPX4 expression, suppress lipid peroxidation, suppress cell death, suppress ROS accumulation, promote proliferation, and prolong survival in tumors. The results provided in this example demonstrates that second organelle complexes (Q) transplantation suppresses ferroptosis in T cells and further demonstrate its utility in cell therapy.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or”unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of producing a population of T cells for adoptive T cell therapy, comprising:
contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus.

2. The method of claim 1, wherein the method comprises stimulating the population of T cells.

3. The method of claim 2, wherein stimulating step expands the population of T cells.

4. A method of enhancing the proliferation, migration, persistence and/or activity of a population of T cells for adoptive T cell therapy, comprising:
contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus; and
stimulating the population of T cells to expand the population of T cells.

5. A method of generating a population of T cells resistant to exhaustion, comprising:
contacting isolated organelle complexes with a population of T cells to generate a population of T cells comprising the organelle complexes, wherein the organelle complexes comprise mitochondria and one or more of endoplasmic reticulum, peroxisomes, lysosomes, and Golgi apparatus; and
stimulating the population of T cells to expand the population of T cells.

6. The method of any one of claims 2-5, wherein the stimulating is performed before the contacting, after the contacting, and/or during contacting.

7. The method of any one of claims 2-6, wherein the stimulating comprises culturing the population of T cells in the presence of one or more stimulating agents.

8. The method of any one of claims 2-7, wherein the one or more stimulating agents comprise an agent that stimulates a CD3/TCR complex-associated signal and an agent that stimulates a costimulatory molecule on the surface of the T cells.

9. The method of any one of claims 2-8, wherein the one or more stimulating agents comprise:
a molecule that binds CD28, optionally one or more of an anti-CD28 antibody, CD80, and CD86; and/or
a molecule that binds CD3, optionally an anti-CD3 antibody.

10. The method of any one of claims 1-9, wherein the stimulating and/or contacting is performed for a period time of at least about 6 hours, 12 hours, 16 hours, 1 day, 2 days, 3 days, 4days, 5 days, 6 days, or 7 days.

11. The method of any one of claims 1-10, wherein the method comprises introducing a heterologous nucleic acid encoding a chimeric antigen receptor (CAR) and/or an engineered T cell receptor (TCR) into the T cells, optionally the introducing step is performed before the contacting, after the contacting, and/or during the contacting, further optionally the heterologous nucleic acid is a vector.

12. The method of any one of claims 1-11, wherein upon contact of the isolated organelle complexes with a population of T cells, the organelle complexes are capable of incorporating into the T cells.

13. The method of any one of claims 1-11, wherein the contacting step is repeated at least 2, 3, or 4 times, optionally the contacting is performed during one or both of the stimulating step and the introducing step.

14. The method of any one of claims 1-13, wherein the population of T cells is contacted with an effective amount of isolated organelle complexes sufficient to enhance the proliferation, migration, persistence and/or activity of said T cells, optionally the effective amount comprises about 20 ug of isolated organelle complexes.

15. The method of any one of claims 1-14, wherein the population of T cells is contacted with an effective amount of isolated organelle complexes sufficient to render the said T cells resistant to exhaustion.

16. The method of any one of claims 1-15, wherein the population of T cells is derived from lymph node cells, optionally purified by a magnetic particle-based enrichment procedure selected from the group consisting of manual MACS®, AutoMACS®, CliniMACS®, EasySep®, and RoboSep®.

17. The method of any one of claims 1-16, wherein the population of T cells comprises one or more of a CD4⁺ cell, a CD8⁺ T cell, a cytotoxic T cell, a terminal effector T cell, an effector T cell, a memory or central memory T cell, a naive T cell, a regulatory T cell, a natural killer T cell, a gamma-delta T cell, a cytokine induced killer (CIK) T cell and a tumor infiltrating lymphocyte (TIL).

18. The method of any one of claims 1-17, wherein the organelle complexes comprise first organelle complexes, second organelle complexes, or a combination of first organelle complexes and second organelle complexes,
wherein the first organelle complexes and second organelle complexes are depleted of cytosolic macromolecules,
wherein first organelle complexes are derived from (i) frozen cells; (ii) floating cells; and/or (iii) cells contacted with a surfactant at a concentration at or above the critical micellar concentration (CMC) for the surfactant, and
wherein second organelle complexes are derived from (i) adherent cells; and/or (ii) cells contacted with a surfactant at a concentration below the critical micellar concentration (CMC) for the surfactant.

19. The method of claim 18, wherein the cytosolic macromolecules comprise cytosolic proteins, wherein the abundance of one or more cytosolic proteins is depleted by at least about 90% as compared to the cells from which the organelle complexes population are derived, optionally the cytosolic proteins are p70S6K and/or glyceraldehyde 3-phosphate dehydrogenase (GAPDH).

20. The method of any one of claims 1-19, wherein the organelle complexes comprise:
one or more mitochondrial matrix proteins, optionally mitochondrial transcription factor A (TFAM) and/or citrate synthase (CS);
one or more outer mitochondrial membrane proteins, optionally outer mitochondrial membrane complex subunit 20 (TOMM20);
one or more lysosome proteins, optionally lysosomal-associated membrane protein 2 (LAMP2), mannose-6-phosphate receptor (M6PR), and/or lysosomal-associated membrane protein 1 (LAMP1);
one or more peroxisome proteins, optionally catalase and/or ATP-binding cassette transporter 1, subfamily D, type 3 (ABCD3);
one or more Golgi apparatus proteins, optionally Golgin-97, Sintaxin-6, TGOLN2/trans-Golgi network protein 2 (TGN46), Golgi matrix protein 130 (GM130), and/or Mannosidase Alpha Class 2A Member 1 (MAN2A1); and/or
one or more endoplasmic reticulum proteins, optionally Calreticulin and/or Calnexin.

21. The method of any one of claims 1-20, wherein the organelle complexes are derived from cells treated with a mitochondria-activating agent, optionally resveratrol.

22. The method of any one of claims 1-21, wherein the organelle complexes are derived from cells of a subject different from the subject from which the T cells are derived.

23. The method of any one of claims 1-22, wherein the organelle complexes are derived from cells of the same subject from which the T cells are derived.

24. The method of any one of claims 1-23, wherein the T cell comprises a chimeric antigen receptor (CAR) and/or an engineered T cell receptor (TCR).

25. The method of any one of claims 1-24, wherein the CAR and/or TCR comprises one or more of an antigen binding domain, a transmembrane domain, and an intracellular signaling domain.

26. The method of claim 25, wherein the intracellular signaling domain comprises a primary signaling domain, a costimulatory domain, or both of a primary signaling domain and a costimulatory domain.

27. The method of claim 26, wherein the primary signaling domain comprises a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof.

28. The method of any one of claims 26-27, wherein the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS (CD278), lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100(SEMA4D), CD69, SLAMF6(NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

29. The method of any one of claims 25-28, wherein the antigen binding domain binds a tumor antigen, optionally the tumor antigen is a solid tumor antigen.

30. The method of any one of claims 25-29, wherein the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising anticomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

31. The method of any one of claims 25-30, wherein the antigen binding domain is connected to the transmembrane domain by a hinge region.

32. The method of any one of claims 25-31, wherein the transmembrane domain comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80(KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof.

33. The method of any one of claims 24-32, wherein the CAR or TCR further comprises a leader peptide.

34. The method of any one of claims 24-33, wherein the TCR further comprises a constant region and/or CDR4.

35. The method of any one of claims 1-34,
wherein T cell recovery efficiency is at least about 5 percent greater as compared to a method that does not comprise contacting isolated organelle complexes with the population of T cells, and
wherein T cell recovery efficiency is the ratio of T cells recovered after stimulation to the number of T cells immediately before the start of stimulation, optionally about 48 hours of stimulation.

36. The method of any one of claims 1-35, wherein the population of T cells exhibit one or more of enhanced expansion capability, enhanced cytotoxicity against target cells, enhanced resistance to exhaustion, and enhanced persistence, as compared to a population of T cells that do not comprise exogenous organelle complexes, optionally in vivo and/or in vitro.

37. The method of any one of claims 1-36, wherein the population of T cells exhibit an at least about 1.1-fold increase in basal and/or maximal oxygen consumption rate as compared to a population of T cells that do not comprise exogenous organelle complexes, optionally in vivo and/or in vitro.

38. The method of any one of claims 1-37, wherein the population of T cells exhibit an at least about 1.1-fold increase in basal and/or maximal glycolytic rate as compared to a population of T cells that do not comprise exogenous organelle complexes, optionally in vivo and/or in vitro.

39. The method of any one of claims 1-38, wherein the population of T cells exhibit an at least about 1.1-fold increase in glycolytic capacity and/or respiratory capacity as compared to a population of T cells that do not comprise exogenous organelle complexes, optionally in vivo and/or in vitro.

40. The method of any one of claims 1-39, wherein the population of T cells exhibit an at least about 1.1-fold increase in mitochondrial mass as compared to a population of T cells that do not comprise exogenous organelle complexes, optionally in vivo and/or in vitro.

41. The method of any one of claims 1-40, wherein the population of T cells exhibit an at least about 1.1-fold increase in cytotoxic activity against target cells as compared to a population of T cells that do not comprise exogenous organelle complexes, optionally in vivo and/or in vitro.

42. The method of any one of claims 1-41, wherein the population of T cells exhibit an at least about 1.1-fold decrease in the level of one or more exhaustion markers as compared to a population of T cells that do not comprise exogenous organelle complexes, optionally the exhaustion markers are selected from the group comprising PD-1, CTLA-4, TIM-3, LAG-3, BTLA, 2B4, CD 160, CD39, VISTA, TIGIT, or any combination thereof, optionally in vivo and/or in vitro.

43. The method of any one of claims 1-42, wherein the population of T cells exhibit an at least about 1.1-fold reduction in levels one or more of cellular ROS, mitochondrial ROS, cellular oxidative stress, and mitochondrial oxidative stress, as compared to a population of T cells that do not comprise exogenous organelle complexes, optionally in vivo and/or in vitro, further optionally the reactive oxygen species comprise superoxide (O₂·⁻), hydroperoxy (HO·₂), hydrogen peroxide (H₂O₂), peroxynitrite (ONOO⁻), hypochlorous acid (HOCl), hypobromous acid (HOBr), hydroxyl radical (HO·), peroxy radical (ROO·), alkoxy radical (RO·), singlet oxygen (¹O₂), lipid peroxides, lipid peroxyradicals or lipid alkoxyl radicals, or any combination thereof.

44. The method of any one of claims 1-43, wherein the population of T cells exhibit an at least about 1.1-fold increase production of one or more of cytokines as compared to a population of T cells that do not comprise exogenous organelle complexes, optionally in vivo and/or in vitro, further optionally the cytokines comprise interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ).

45. The method of any one of claims 1-44, wherein the population of T cells exhibit an at least about 1.1-fold increase in cell proliferation as compared to a population of T cells that do not comprise exogenous organelle complexes, optionally in vivo and/or in vitro.

46. The method of any one of claims 1-45, wherein the population of T cells exhibit an at least about 1.1-fold increase in cell viability following chronic TCR stimulation as compared to a population of T cells that do not comprise exogenous organelle complexes, optionally in vivo and/or in vitro.

47. A population of T cells generated by the method of any one of claim 1-46.

48. A pharmaceutical composition, comprising:
a population of T cells generated by the method of any one of claim 1-46; and
one or more pharmaceutically acceptable carriers.

49. A population of T cells for an adoptive T cell therapy,
wherein the T cells comprise exogenous organelle complexes,
wherein the organelle complexes comprise mitochondria and one or more of endoplasmic reticulum, peroxisomes, lysosomes, and Golgi apparatus, and
wherein the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

50. A method of treating or preventing a disease or disorder in a subject, comprising:
administering to the subject an effective amount of the population of T cells generated according to the method of any one of claim 1-46, thereby treating or preventing the disease or disorder in the subject,
wherein the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

51. A method for enhancing adoptive T cell therapy in a subject, comprising:
generating an effective amount of T cells comprising exogenous organelle complexes according to the method of any one of claim 1-46; and
adoptively transferring said T cells to the subject,
wherein the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

52. A method of treating or preventing a disease or disorder in a subject, comprising:
administering to the subject an effective amount of a population of T cells, optionally said T cells comprise a chimeric antigen receptor (CAR) and/or an engineered T cell receptor (TCR); and
administering to the subject an effective amount of isolated organelle complexes, thereby treating or preventing the disease or disorder in the subject,
wherein the isolated organelle complexes are capable of contacting the population of T cells in vivo to generate a population of T cells comprising the organelle complexes,
wherein the population of T cells exhibit one or more of enhanced expansion capability, enhanced cytotoxicity against target cells, enhanced resistance to exhaustion, and enhanced persistence, as compared to a population of T cells that do not comprise exogenous organelle complexes.

53. The method of claim 52,
wherein the subject is administered an effective amount of the population of T cells prior to administering to the subject an effective amount of isolated organelle complexes;
wherein the subject is administered an effective amount of isolated organelle complexes prior to administering to the subject an effective amount of the population of T cells; or
wherein the subject is administered an effective amount of the population of T cells and an effective amount of isolated organelle complexes simultaneously.

54. A method of treating or preventing a disease or disorder in a subject, comprising:
administering to the subject an effective amount of a heterologous nucleic acid encoding a chimeric antigen receptor (CAR) and/or an engineered T cell receptor (TCR), optionally the heterologous nucleic acid is a vector, further optionally a viral vector; and
administering to the subject an effective amount of isolated organelle complexes, thereby treating or preventing the disease or disorder in the subject,
wherein the heterologous nucleic acid and isolated organelle complexes are capable of contacting T cells of the subject in vivo to generate T cells comprising the organelle complexes and a CAR and/or an engineered TCR, and
wherein said T cells exhibit one or more of enhanced expansion capability, enhanced cytotoxicity against target cells, enhanced resistance to exhaustion, and enhanced persistence, as compared to T cells that do not comprise exogenous organelle complexes.

55. The method of any one of claims 1-46 or 50-54, wherein the in vivo persistence of the population of T cells comprises a period of about 15 days, about 30 days, about 60 days, about 30 days, or about a year.

56. The method of any one of claims 1-46 or 50-55, wherein the population of T cells reduces tumor volume, tumor growth, and/or tumor burden in the subject, optionally the population of T cells reduces tumor volume in the subject at least about 1.1-fold as compared with the tumor volume in an untreated subject or a subject administered a population of T cells that do not comprise exogenous organelle complexes.

57. The method of any one of claims 1-46 or 50-56, wherein the population of T cells increases overall survival or progression-free survival, optionally the population of T cells increases overall survival or progression-free survival at least about 1.1-fold as compared with untreated subjects or subjects administered a population of T cells that do not comprise exogenous organelle complexes.

58. The method of any one of claims 1-46 or 50-57, wherein the T cells are autologous to the subject.

59. The method of any one of claims 1-46 or 50-58, wherein the T cells are allogenic to the subject.

60. The method of any one of claims 1-46 or 50-59, wherein the adoptive T cell therapy is a CAR-T cell therapy.

61. The method of any one of claims 1-46 or 50-60, wherein the adoptive T cell therapy is an engineered TCR-T cell therapy.

62. The method of any one of claims 1-46 or 50-61, wherein the adoptive T cell therapy is a tumor infiltrating lymphocyte (TIL) therapy.

63. The method of any one of claims 50-62, wherein the administering comprises administering at least about 1×106 T cells, optionally intravenously.

64. The method of any one of claims 50-63, wherein the method comprises repeated administrations of the population of T cells.

65. The method of any one of claims 50-64, wherein the subject is a mammal.

66. The method of any one of claims 50-65, wherein the disease or disorder is associated with expression of a tumor antigen, and wherein the disease associated with expression of a tumor antigen is selected from the group consisting of a proliferative disease, a precancerous condition, a cancer, and a non-cancer related indication associated with expression of the tumor antigen.

67. The method of claim 66, wherein the cancer is selected from the group consisting of colon cancer, rectal cancer, renal-cell carcinoma, liver cancer, small cell or non-small cell carcinoma of the lung, mesothelioma, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers, combinations of said cancers, and metastatic lesions of said cancers.

68. The method of any one of claims 66-67, wherein the cancer is a hematologic cancer chosen from one or more of chronic lymphocytic leukemia (CLL), acute leukemias, acute lymphoid leukemia (ALL), B-cell acute lymphoid leukemia (B-ALL), T cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

69. The method of any one of claims 50-68, wherein administering comprises systemic administration, intrathecal administration, intracranial injection, aerosol delivery, nasal delivery, vaginal delivery, rectal delivery, buccal delivery, ocular delivery, local delivery, topical delivery, intracistemal delivery, intraperitoneal delivery, oral delivery, intramuscular injection, intravenous injection, subcutaneous injection, intranodal injection, intratumoral injection, intraperitoneal injection, intradermal injection, or any combination thereof, optionally the systemic administration is intravenous, intramuscular, intraperitoneal, or intraarticular.
